(19) United States
(12) Patent Application Publication
(10) Pub. No.: US 2024/0170113 A1
(43) Pub. Date: May 23, 2024

(54) VERIFIED SECURE BIOMETRIC COLLECTION SYSTEM

(71) Applicant: SCHNEIDER ADVANCED BIOMETRIC DEVICES CORP., Wilmington, DE (US)

(72) Inventors: David Lyle SCHNEIDER, Hong Kong (CN); MARTIN ATWOOD, CANON CITY, CO (US); Holly Ying Li, New York, NY (US)

(21) Appl. No.: 18/528,303

(22) Filed: Dec. 4, 2023

Related U.S. Application Data
(63) Continuation-in-part of application No. 17/662,830, filed on May 10, 2022.
Application No. 17/662,830 is a continuation-in-part of application No. 16/821,990, filed on Mar. 17, 2020, now Pat. No. 11,361,852.
Application No. 16/821,990 is a continuation of application No. PCT/US18/44823, filed on Aug. 1, 2018.
Application No. PCT/US18/44823 is a continuation of application No. 15/707,431, filed on Sep. 18, 2017, now Pat. No. 10,468,129.
(60) Provisional application No. 62/395,514, filed on Sep. 16, 2016.

(51) Int. Cl. G16H 10/60; G06F 21/32; G06F 21/62; G06F 21/64; H04L 9/06; H04L 9/08; H04L 9/32

(52) U.S. Cl. CPC G16H 10/60; G06F 21/32; G06F 21/6245; G06F 21/64; H04L 9/06; H04L 9/0866; H04L 9/0894; H04L 9/3231; H04L 2209/88

(57) ABSTRACT

A verified secure biometric collection system and method for collecting and recording data from a user associated with a secure transaction, includes a storage device, user input interface, camera, biometric scanner, camera encoder to combine/hash the user image into combined data sets that are stored in the storage device, a biometric encoder to process the biometric scan into biometric template data sets and store them in the storage device. The system includes at least one of a human witness and non-human digital witness, the non-human digital witness having access to sensor inputs and software, to verify that the user is a person, and a privacy encoder to combine and encrypt the data sets into encrypted data sets memorializing the secure transaction. The system produces a secure transaction copy including encrypted data sets, and purges of all the user data collected once the copy of the secure transaction is produced.

### CROSS-REFERENCE TO RELATED APPLICATIONS

This continuation-in-part application claims the benefit and priority of U.S. patent application Ser. No. 17/662,830, titled “Secure Biometric Collection System,” filed May 10, 2022, which is a continuation-in-part application of U.S. patent application Ser. No. 16/821,990, titled “Collecting Apparatus and Method,” filed Mar. 17, 2020, now U.S. Pat. No. 11,361,852 issued on Jun. 14, 2022, which is a continuation of PCT International Application No. PCT/US18/44823, titled “Collecting Apparatus and Collecting Method, filed Aug. 1, 2018 in the U.S. Receiving Office of the U.S. Patent and Trademark Office, which is a continuation of U.S. patent application Ser. No. 15/707,431, titled “Biometric Medical Antifraud and Consent System,” filed on Sep. 18, 2017, which claims priority to U.S. provisional patent application Ser. No. 62/395,514, filed on Sep. 16, 2016, and incorporates by reference the disclosures of which are incorporated by reference herein in their entireties.

### BACKGROUND

### 1. Field of the Invention

The present disclosure generally relates to the protection of personally identifiable data, and more particularly, the present disclosure relates to a secured and verified collecting apparatus and method for reading, encoding, recording, verifying and protecting biometric or non-biometric data.

### 2. Related Art

Modern society has increasingly become a digital based society that increasingly utilizes computer networks to collect, transmit, and process personal data and information from individuals in the society. This personal data and information may be related to, for example, business, employment, private financial and/or medical information and records. Recently, wide range of artificially generated avatars mimicking facial, voice or other biometrics of a human client have become increasingly available in the market place. These avatars or voice robots are used in wide range of commercial or non-commercial transactions, such as banking, healthcare, brokerage trading, governmental organizations, artistic generations, educational institutions and other streams of commerce or society. And are primarily acting as proxies to the respective senior party.

Increasingly, people have been working remotely from their regular places of business, participating in telemedicine doctor's appointments, banking online, educations, and even participated in legal court proceedings remotely. As a result of all these types of activities, there has been a rising challenge to maintain security, privacy, originality and truthfulness of the image or voice behind a purported client; and assure that proper consent is present for these activities conducted by an artificial intelligence (AI) generated representative moderating the transaction at hand.

Unfortunately, this increased digitally and AI generated activity has also increased the activity of hackers who are actively attempting to obtain the secure and private data and information by using AI generated avatars voice robots, as imposters. These hackers may be motivated by personal, political, nation-state and/or economic objectives. As result, people and business entities are concerned about the safety and integrity of their data and information. For example, in the medical industry, patients have a heightened concern about the privacy, accuracy and disclosure of their sensitive health-related information. People are also concerned that their passwords, bank accounts, and credit cards may be vulnerable to these types of hackers who use AI generated imposters to obtain their private information without boundaries. Further, increasing amount of AI generated artistic, academic, educational, or other creative works that appeared to be proprietary are not necessarily the creation of the authors. Hence, increasing concern of originality is prevalent alongside of AI's pervasiveness in our society.

Current industry trends tend to address these problems with generic security solutions focused on applications, databases, firewalls and activity alarm systems. For example, known approaches for attempting to address these problems may include proprietary encryption of manufactured storage devices installed in servers and workstations to protect against unauthorized disclosure. However, even with these approaches, significant data breaches have still occurred, and will likely continue to occur. Verification tools that screen content at the user or viewer end is another approach to address the fidelity and origination of creative works. However, these solutions are not designed to address the issues at the generation end, before the AI-impostered information being disseminated or transmitted to users. Therefore, they are not effective and often being too late after a user already revealed confidential and proprietary information. This has become a serious public problem that needs to be addressed urgently.

Recently, along with the development of blockchain technologies, industry leaders have started to look into more effective ways to cure the aforementioned problem with this immutable, transparent and decentralized data gatekeeping process. Blockchain is a form of distributed ledger technology (DLT) that allows digitized information such as cryptocurrencies to move freely from one user to another who are located on the same network without the involvement of a central/intermediatory party, such as a central bank. In reality, Blockchain functions as an electronic database, referred to as immutable ledger by some, enables users to keep important information secured and unalterable. Records on a blockchain cannot be changed. Only new data blocks can be created to memorialize any new data input. See https://www.techtarget.com/searchitoperations/tip/Blockchain-An-immutable-ledger-to-replace-the-database. Although blockchain has initially been developed in the financial industry along with transactions of crypto currencies such as Bitcoin or Ethereum, blockchain can also be implemented in many other industries. However, there are still serious lack of effective blockchain implementations in wide range of industries, such as airport security, police department, medical or public health systems, especially along with security, authentication and antifraud detections.

### SUMMARY OF THE INVENTION

In one aspect of an exemplary embodiment, disclosed is a Verified Secure Biometric Collection (VSBC) system for collecting, verifying and recording data from a user associated with a secure transaction. The VSBC system comprises a storage device, input device, collecting device such as a camera, a voice recorder or a biometric scanner; encoding devices such as a camera encoder, a voice encoder, or biometric sensor, or fingerprint encoder; a verification device for verifying and stamping; and a privacy encoder. The input device is configured to receive information from the user related to the secure transaction, the camera is configured to capture an image of the user and store it in the storage device, the voice recorder is configured to record the voice of the user and store it in the storage device; and the biometric (fingerprint, etc.) scanner is configured to scan biometric (fingerprint, etc.) information of the user and store it in the storage device. The camera encoder is configured to combine and hash the image, voice recording or biometric (e.g., fingerprint, etc.) scan of the user into combined data sets that are stored in the storage device and the image, voice or biometric encoder is configured to process the image, voice or biometric scan of the user into image, voice or biometric template data sets and store the template data sets in the storage device. The verification device is configured to verify a human or an artificial intelligence (AI) generation of the data sets and stamp it with a symbol after a human witness (or digital witness) verifies and sends a verification signal and stamp. The system may be configured to produce a copy of the image, voice or biometric data of the user together with a verification stamp generated from the physical or digital witness. The privacy encoder is configured to combine and encrypt the combined data sets and image, voice or biometric template data sets from the storage device into encrypted data sets that memorialize the secure transaction. The system is configured to produce a copy of the secure transaction that includes the encrypted data sets of the user and the storage device is configured to be purged of all the data collected from the user including the information from the user and all biometric and non-biometric data that includes the image, voice or fingerprint of the user's combined and template data sets, and encrypted data sets once the copy of the secure transaction is produced. The VSBC system may be further configured to allow the produced copy of the image, voice or biometric data of the user into an immutable ledger or a blockchain structure thereby maintaining a history of transactions that are secured, irreversible and decentralized. It should be noted that in the event the user is an AI generated persona, the biometric data could be image or voice at the current state of the technical development. However, when artificial skin and sensory technology reaches the level of authenticity, the fingerprint biometric information can also be gathered from an AI generated persona.

In another aspect of an exemplar embodiment, disclosed is a VSBC system that comprises one or more processing units and a computer-readable medium having encoded thereon computer-executable instructions. The computer-executable instructions cause the one or more processing units to: receive information from the user related to the secure transaction with an input device, where the received information from the user is stored in a storage device or an immutable ledger, capture an image, voice or fingerprint (biometric/non-biometric data) of the user with a camera, voice recorder or finger print scanner where the image, voice or fingerprint is stored in the storage device or the immutable ledger, where the image, voice or fingerprint information of the user is transmitted to a human (or digital) witness for verification whereby a “verification stamp” as an optional additional security measure. For example, a visible marker such as “HIS or AIS” attached with an industry-standard PKI certificate which communicates feedback to web browser viewers and visual feedback trust stamp, or equivalent symbols representing verification conventions in industry are generated; combine and hash the verified and stamped image, voice or fingerprint (or biometric/non-biometric) information of the user into combined data sets that are stored in the storage device or the immutable ledger, process the verified and stamped image, voice or fingerprint (biometric or non-biometric information) of the user into a set of verified and stamped biometric and non-biometric information template data sets that are stored in the storage device or the immutable ledger, combine and encrypt the combined data sets and verified and stamped biometric or non-biometric template data sets from the storage device into encrypted data sets memorializing the secure and verified transaction, produce a copy of the secure transaction that includes the encrypted data sets for the user, or upload the copy of the secure transaction with the encrypted data sets to the immutable ledger at the user's choice or at an institution's mandate, and purge all the data collected from the user including the information from the user and all biometric and non-biometric data that includes the image, voice or fingerprint of the user, combined data sets, verified and stamped biometric and non-biometric information template data sets, and encrypted data sets once the copy of the secure transaction is produced and transmitted to an intended user.

In another aspect of an exemplary embodiment, the VSBC system performs a method that comprises: receiving information from the user related to the secure transaction with an input device; storing the received information from the user in a storage device or an immutable ledger; capturing information such as an image, voice or fingerprint of the user with a camera or scanning device; storing the captured information in the storage device or the immutable ledger; scanning an image, voice or fingerprint of the user with an image, voice or fingerprint scanner; storing the image, voice or fingerprint of the user in the storage device or the immutable ledger; verifying the image, voice or fingerprint of the user with a verification witness (or artificial proxy); stamping the verified image, voice or fingerprint information with a verification stamp as an optional additional security measure, such as for example, HIS or AIS or equivalent (these symbols representing verifications conventions in industry); combining and hashing the verified and stamped information of the user into combined data sets; storing the combined data sets in the storage device or the immutable ledger; processing the verified and stamped image, voice or fingerprint scan of the user into template data sets; storing the verified and stamped template data sets in the storage device or the immutable ledger; combining and encrypting the combined data sets and verified and stamped template data sets from the storage device into encrypted data sets memorializing the verified and secured transaction; producing a copy of the verified and secured transaction that includes the encrypted data sets for the user; and uploading the copy of the verified and secured transaction with the encrypted data sets to the immutable ledger at the user's choice before purging all the data collected from the user including the information from the user that includes the image, voice or fingerprint of the user, combined data sets, verified and stamped image, voice or fingerprint template data sets, and encrypted data sets once the copy of the secure transaction is produced.

In yet another aspect of an exemplary embodiment, a VSBC system for collecting and recording data from a user associated with a secure transaction is provided, comprising: a storage device; an input interface configured to receive information from the user related to the secure transaction; a camera configured to capture an image of the user; a biometric scanner configured to scan a biometric of the user; a camera encoder configured to combine and hash the image of the user into combined data sets that are stored in the storage device; a biometric encoder configured to process the biometric scan of the user into biometric template data sets and store the biometric template data sets in the storage device; at least one of a human witness and non-human digital witness, the non-human digital witness having access to sensor inputs and software, configured to verify that a person providing at least one of the image capture and biometric scan is the user, wherein the at least one human witness and non-human digital witness is in proximity to the user; and a privacy encoder configured to combine and encrypt the combined data sets and biometric template data sets from the storage device into encrypted data sets memorializing the secure transaction, wherein the VSBC system is configured to produce a copy of the secure transaction that includes the encrypted data sets for the user, and wherein the storage device is configured to be purged of all the data collected from the user including the information from the user and all biometric data that includes the image of the user, biometrics of the user, combined data sets, biometrics template data sets, and encrypted data sets once the copy of the secure transaction is produced.

In yet another aspect of an exemplary embodiment, the above system is described, further including a second camera, wherein the first camera is configured to capture the image of the user in a visible range, the second camera is configured to capture a second image of the user in a non-visible range, and the camera encoder is configured to combine and hash the image of the user in the visible range and the image of user in the non-visible range into the combined data sets; and/or further configured to transmit the data collected from the user including the information from the user and all biometric data that includes the image of the user, the biometric measurement of the user, combined data sets, biometric template data sets, and encrypted data sets once the copy of the secure transaction is produced to an immutable distributed ledger upon receiving the user's instruction; and/or wherein the sensor inputs are one or more witness-cameras in proximity to the user, having image processing software to assist in the verification; and/or wherein the sensor inputs further comprises, at least one of a microphone, speaker, thermal sensor, and display, with additional processing software to assist in the verification; and/or wherein the non-human digital witness comprises an artificial intelligence engine, operating to analyze sensor data taken of the user to verify the user is an actual person; and/or further comprising an avatar as an interface for the artificial intelligence engine with the user; and/or wherein the artificial intelligence engine works in concert with data from at least one of the input interface, camera, and biometric scanner for verification of the user; and/or further including a processor, wherein the biometric encoder is further configured to timestamp the biometric scan of the user with a first timestamp and a verification indication from the non-human digital witness with a second timestamp, and the processor is configured to determine a difference between the first timestamp and second timestamp is less than or equal to a predetermined time; and/or wherein the storage device is in a network Cloud and the input interface operates as a live encoded interface; and/or further comprising, a visual token code produced from the privacy encoder and assigned to one or more captured images of the user; and/or further comprising: a cryptographic hash Audit Token data element, the data element containing at least one of transaction metadata, identification recordings, and verification of identification recordings; and an encrypted Audit Token from encrypting the data element, wherein the encrypted Audit Token provides access to a connected Cloud-based data store; and/or further comprising, an Audit Token and a common-format PDF Receipt, wherein the Audit Token is used to inform an external system of confirmation and recording of the transaction; and/or further comprising, a certificate document generated from the transaction, containing an encrypted user identity token; and/or further comprising, a non-repudiable, one-time transaction encoding of details of the transaction, containing user identity tokens in plurality for a plurality of users; and/or wherein said user is at least one of a human or artificial intelligence created persona.

In yet another aspect of an exemplary embodiment, a method of collecting and recording data from a user associated with a secure transaction is provided, comprising: receiving information from a user via an input interface, relating to the secure transaction; capturing an image of the user; scanning a biometric of the user; electronically combining and hashing the image of the user into combined data sets for storage; electronically processing the biometric scan of the user into biometric template data sets for storage; witnessing the user via at least one of a human witness and non-human digital witness, in proximity to the user; providing a verification of the user via the witnessing; electronically combining and encrypting the combined data sets and biometric template data sets from storage into encrypted data sets memorializing the secure transaction; electronically producing a private copy of the secure transaction including the encrypted data sets for the user; and purging from storage all the data collected from the user including the information from the user and all biometric data that including the image of the user, biometrics of the user, combined data sets, biometrics template data sets, and encrypted data sets once the copy of the secure transaction is produced.

In yet another aspect of an exemplary embodiment, the above method is provided, further comprising, capturing an image of the user in a non-visible range, wherein image data of the non-visible range is also combined and hashed into the combined data sets; and/or further comprising, the system is further configured to transmit the data collected from the user including the information from the user and all biometric data that includes the image of the user, the biometric measurement of the user, combined data sets, biometric template data sets, and encrypted data sets once the copy of the secure transaction is produced to an immutable distributed ledger upon receiving the user's instruction; and/or further comprising, providing one or more witness-cameras in proximity to the user to assist in the verification by the non-human digital witness; and/or further comprising, acquiring real-time data of the user via at least one of a microphone, speaker, thermal sensor, and display; and/or further comprising, using an artificial intelligence engine as the non-human digital witness; and/or, further comprising, interfacing with the user via an avatar; and/or further comprising, processing data from the captured image of the user and the scanned biometric of the user by the artificial intelligence engine for verification of the user; and/or further comprising: timestamping the biometric scan of the user with a first timestamp and a verification indication from the non-human digital witness with a second timestamp; and determining a difference between the first timestamp and second timestamp is less than or equal to a predetermined time; and/or further comprising, producing a visual token code and assigning to one or more captured images of the user; and/or further comprising, cryptographically hashing an Audit Token data element containing at least one of transaction metadata, identification recordings, and verification of identification recordings, to form an encrypted Audit Token for accessing a Cloud-based data store; and/or further comprising, informing an external system of confirmation and recording of the transaction via an Audit Token and a common-format PDF Receipt; and/or further comprising, generating a certificate document from the transaction, containing an encrypted identity token; and/or further comprising, encoding details of the transaction in a non-repudiable, one-time transaction format, containing identity tokens in plurality for a plurality of users; and/or further comprising, upon completing the secure transaction, enabling access for the user to at least one of a social media site, restricted access site, government site, financial site, and virtual site; and/or further comprising, enabling at least one of transaction certification, transaction certification, and recording of encrypted transaction tokens; and/or further comprising, electronically notarizing, via the non-human digital witness, the transaction; and/or further comprising, proving physical proximity and spatial geographic-location of the user to the input interface; and/or further comprising, reading and recording biometric data for at least two persons proximal to the user input; and/or further comprising, proving physical proximity and spatial geographic-location of the user to the input interface; and/or further comprising, capturing via a non-visible light capturing camera at least one facial, skin, and distinguishing marks of the user; and/or further comprising, pre-emptively encrypting and privatizing recordings for real-time transmission and storage in a Cloud-based data store; and/or further comprising, receiving confirmation and cryptographic validations, in plurality, from the Cloud-based data store, to determine validity or denial of validity for the recordings; and/or further comprising, transmitting a permanent, timestamped and geographically-stamped Audit Token to an external public cloud archive data store, when geographic location contains at least one of GPS, GLONASS, and terrestrial cellular location data; and/or further comprising, electronically performing a cryptographic non-recoverable erase on data recordings; and/or further comprising, transmitting at least the encrypted data sets and secure transaction into at least one of a Cloud Based Auditing service, Cloud Based recording service, third-party computing system and governmental computing system; and/or further comprising, transmitting a common-format PDF Document certificate into a Cloud Based Data store, containing serialized confirmation specifying user Identity and transaction token recordings, for future verification application; and/or wherein said user is at least one of a human or artificially intelligent persona.

Other devices, apparatuses, systems, methods, features, and advantages of the invention will be or will become apparent to one with skill in the art upon examination of the following figures and detailed description. It is intended that all such additional devices, apparatuses, systems, methods, features, and advantages be included within this description, be within the scope of the invention, and be protected by the accompanying claims.

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 is a system block diagram of an example of an implementation of a VSBC system within a secure network in accordance with the present disclosure.

FIG. 2 is a system block diagram of an example of an implementation of the VSBC system in accordance with the present disclosure.

FIG. 3 is a system block diagram of an example of an implementation of components of the VSBC system, shown in FIGS. 1 and 2, in accordance with the present disclosure.

FIG. 4 is a perspective front view of an example of an implementation of the VSBC system, shown in FIGS. 1 and 2, in accordance with the present disclosure.

FIG. 5 is a flowchart diagram for an example of an implementation of a collecting method performed by the VSBC system, shown in FIGS. 1 and 2, in accordance with the present disclosure.

FIG. 6 is a system block diagram of an example of an implementation of the VSBC system, shown in FIGS. 1 and 2, for a point-of-sale application in accordance with the present disclosure.

FIG. 7 is a system block diagram of an example of an implementation of the VSBC system, shown in FIGS. 1 and 2, in a compact, audio-capture recorder form in accordance with the present disclosure.

FIG. 8 is a flowchart diagram for an example of an implementation of a method for encoding private biometric data into an audit record in accordance with the present disclosure.

FIG. 9 is a flowchart diagram of an example of an implementation of a method for audio-capture recorder logic applied in compact the VSBC system, shown in FIG. 7, in accordance with the present disclosure.

FIG. 10 is a system block diagram of an example of an implementation of a method for exporting audit record to a private encrypted electronic datastore in Cloud, an immutable ledger such as a blockchain and external applications that meet governmental privacy regulations for protection of biometric data in accordance with the present disclosure.

FIG. 11 is a flowchart of an example of an implementation of a method for data capture in accordance with the present disclosure.

FIG. 12 is a system block diagram of an example of an implementation of hardware logic that delivers a guaranteed, tamper proof solid-state mirrored visual display with identical data content in accordance with the present disclosure.

FIG. 13 is a flowchart of an example of an implementation of a data capture method for recording transactions between two parties while recording physical proximity for audit purpose in accordance with the present disclosure.

FIG. 14 is a system block diagram of an example of an implementation of the system bus, shown in FIG. 2, that provides a certified screen capture audit in accordance with the present disclosure.

FIG. 15 is a system block diagram of an example of another implementation of the VSBC system, shown in FIG. 1, within a secure network in accordance with the present disclosure.

FIG. 16 is a flow diagram of one example implementation of the exemplary VSBC system with a service Software-as-a-Service (SaaS) Cloud system.

FIG. 17 is a block/function diagram of a VSBC system with a digital electronic witness or AI persona, in concert with data exchange and/or storage with a Cloud.

### DETAILED DESCRIPTION

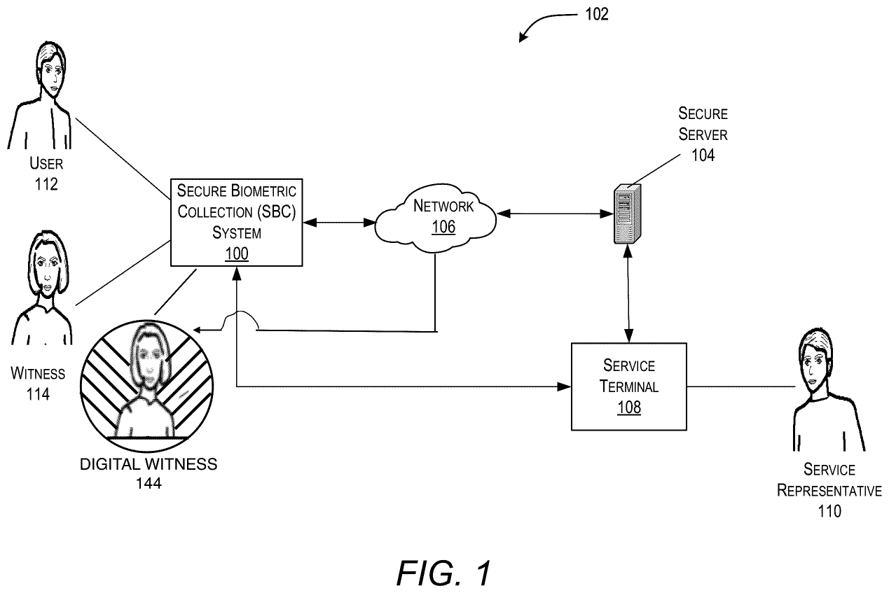

A Verified Secure Biometric Collection (VSBC) system for collecting and recording data from a user associated with a secure transaction is disclosed. In FIG. 1, a system block diagram of an example of an implementation of a VSBC system 100 within a secure network 102 is shown in accordance with the present disclosure. The secure network 102 may include a secure server 104 that communicates with the VSBC system 100 via a network 106. The network 106 may include one or more network such as, for example, an Ethernet network, wireless network, the Internet, a distributed blockchain network or other type of communications network. In this non-limiting example, the service server 104 may include one or more servers utilized by a business or government entity to communicate and control the VSBC system 100. The secure server 104 may also communicate with a service terminal 108 that is utilized by the business or government entity to communicate with the VSBC system 100 via the secure server 104 and network 106. The service terminal 108 may be a computer device, such as a desktop personal computer, laptop computer, or other computer device utilized by a service representative 110 that works for business or government entity. The secure network 102 can also be designed as a decentralized blockchain structure.

In this non-limiting example, the secure network 102 may be secure health network and the business entity may be a health provider that utilizes the VSBC system 100 to obtain health related information from a user 112. The user 112 may be a person seeking medical care that expects his/her personal information to be kept private. In addition to the user 112, a witness 114 may be present and proximal when the user 112 interfaces with the VSBC system 100. As an example, the witness 114 may be a parent of the user 112 if the user 112 is underage. In this non-limiting example, the service representative 110 may be health provider employee that assists the user 112 in processing his/her health care needs. In one example, the VSBC system 100 may be a standalone kiosk in a medical facility. Alternatively, the VSBC system 100 and the service terminal 108 may be the same system that is physically located near the service representative 110. In this non-limiting example, combined VSBC system 100 and service terminal 108 may be located at a check-in location in the medical facility in front of the service representative where the user 112, and optionally the witness 114, approach the service representative 110 and input the user's 112 information at the VSBC system 100 in front of the service representative 110 where the service representative 110 also interfaces directly with the VSBC system 100.

In another example, the secure network 102 may be secure government network and the government entity utilizes the VSBC system 100 to obtain sensitive government related information from a user 112. In this non-limiting example, the government entity may be a government taxing authority, courthouse, central bank or other government related facility.

In yet another example, the secure network 102 may be secure government related network and the business entity utilizes the VSBC system 100 to obtain sensitive government related information from a user 112. In this non-limiting example, the business entity may be a defense contractor or a business the performs services for a government entity.

In still another example, the secure network 102 may be a business-related network and business entity utilizes the VSBC system 100 as a point-of-sale device. In this non-limiting example, the user 112 may be a buyer, the VSBC system 100 may be combined with the service terminal 108 as a point-of-sale device, and the service representative 110 may be a sales representative of the business entity.

As an example of the business entity being a health provider, the VSBC system 100 may be a kiosk (such as, for example VSBC system 400) within the facility of the health provider. The user 112 may then approach and activate the VSBC system 100 to receive medical services. The VSBC system 100 would then prompt the user 112 to input the identify and personal information related to the identity of the user 112 and the requested medical services. For example, is the user 112 requesting to receive medical services for a previously booked medical appointment or walk-in medical services. The user 112 would input this information and any related user data into the VSBC system 100 which would be related to a private and secure transaction with the medical provider that may include financial and private medical information about the user 112. This information would be received by VSBC system 100 and stored in a storage device. The VSBC system 100 may then capture (i.e., take) one or more pictures and/or video of the user 112 with one or more cameras of the VSBC system 100. The captured images or videos would then be stored in the storage device. The VSBC system 100 would then prompt the user 112 for biometric data such as one or more fingerprints from the user 112 with a fingerprint scanner. The user 112 would then scan his/her fingerprint, or fingerprints, with the fingerprint scanner and the VSBC system 100 would store the fingerprint (s) into the storage device. The VSBC system 100 would then combine and hash the image (s) of the user into combined data sets and store the combined data sets into the storage device. The VSBC system 100 would then process the fingerprint scan of the user into fingerprint template data sets and then store the fingerprint template data sets into the storage device. The VSBC system 100 would then combine and encrypt the combined data sets and fingerprint template data sets from the storage device into encrypted data sets memorializing the secure transaction, or transmit the secure transaction and including the encrypted data set to an immutable ledger, and produce a copy of the secure transaction that includes the encrypted data sets for the user 112. The VSBC system 100 would then purge all the data collected from the user including the information from the user and all biometric data that includes the image of the user, fingerprint of the user, combined data sets, fingerprint template data sets, and encrypted data sets once the copy of the secure transaction is produced and/or transmitted to the immutable ledger.

In this non-limiting example, to increase the level of security, the VSBC system 100 may require that a second person to authenticate the information provided by the user 112. The second person may be the witness 114 or the service representative 110.

As an example, the user 112 may be a patient that is requiring medical services and the VSBC system may collect the information from the user 112 and then present an agreement document for the user 112 to read before services are rendered by the health provider. The agreement may be displayed on display device on the VSBC system 100 for the user 112 to read. The VSBC system 100 then would prompt the user 112 to accept or decline the agreement document. If the user 112 does not accept the agreement document, the v system 100 then ends the session with the user 112 and resets for another user. If, instead, the user 112 does accept the agreement document, the VSBC system prompts the user 112 to be photographed and/or video recorded. This prompt may be timed by a first predetermined time (for example, three to five seconds) for the user 112 to prepare to be photographed and/or video recorded by the camera. Once photographed and/or recorded, the VSBC system 100 will prompt the user 112 to provide one or more fingerprints via the fingerprint scanner of the VSBC system 100. Once the user 112 provides the fingerprint (s), the VSBC system 100 may prompt the second person to also provide a fingerprint via the same or another fingerprint scanner. In this situation, the second person may be the witness 114 that is proximate to the user 112 to confirm the acceptance of agreement document by the user 112 or the service representative 110 that may be proximate or remote from the user 112. In the situation where the VSBC system 100 is a kiosk (combining the VSBC system 100 and service terminal 108) that is close to the service representative 110, the service representative 110 may provide the fingerprint scan while personally viewing and being personally close to the user 112. In the situation where the VSBC system 100 is a kiosk that is remote from the service terminal 108 and service representative 110, the service terminal 108 may include a display that shows live video of the user 112 at the VSBC system 100, where the video is recorded by a camera of the VSBC system 100. In this situation, the service representative 110 may provide a fingerprint to the VSBC system 100 via a fingerprint scanner on the service terminal that scans the fingerprint of the remote service representative 110 and transmits the scanned fingerprint of the service representative 110 to the VSBC system 100 via the service terminal 108, secure server 104, and network 106.

In these examples, the VSBC system 100 may time how long it takes for the second person to provide the fingerprint of the second person to ensure that it is approximately contemporaneous and provided as soon as possible after the user 112 provided the scanned fingerprint. As such, the VSBC system 100 may require that the second person's fingerprint is scanned within a second predetermine time, for example about 1000 ms, after the user's 112 fingerprint is scanned. The VSBC system 100 determines if the second person's fingerprint was scanned within the second predetermine time by timestamping the user's 112 scanned fingerprint and the timestamping the second person's scanned fingerprint. If the difference between the first timestamp and the second timestamp is within the second predetermined time, the VSBC system 100 will accept the second person's fingerprint. If not, the VSBC system 100 may require that the user 112 and second person repeat the process of scanning their respective fingerprints until they do it within the second predetermined time.

Once the VSBC system 100 accepts that both the user's 112 and the second person's fingerprints were provided within the second predetermined time, the VSBC system 100 would then combine and hash the image (s) of the user into combined data sets and store the combined data sets into the storage device. The VSBC system 100 would then process the fingerprint scans of the user 112 and second person into fingerprint template data sets and then store the fingerprint template data sets into the storage device. The VSBC system 100 would then combine and encrypt the combined data sets and fingerprint template data sets from the storage device into encrypted data sets memorializing the secure transaction, produce a copy of the secure transaction that includes the encrypted data sets for the user 112 and/or transmit the copy of the secure transaction to an immutable ledger located in the same network. The VSBC system 100 would then purge all the data collected from the user including the information from the user and all biometric data that includes the image of the user, fingerprint of the user, fingerprint of the second person, combined data sets, fingerprint template data sets, and encrypted data sets once the copy of the secure transaction is produced or being transmitted to the immutable ledger.

In another example, the witness 114 may not be a physical person but an automated non-human entity 144, for example a digital witness or avatar recognized by the VSBC system 100 as a trusted information source acting as the witness 114, and if so desired interacting with the user 112 to accomplish its digital witnessing function. The automated entity 144 serves, via “electronic sensor” means, to function as the witness 114. The automatic entity 144 could take the form of a separate data gathering device or system, electronically “verifying” user 112. This “verifying” can take the form of tracking one or more of the interactions of the user 112 with the VSBC system 100 so as to determine that user 112 is indeed the same acting person by, for example, time-comparing or motion-comparing the user's 112 responses to the VSBC system's 100 prompts. This “verification” of a user 112 as a human actor can be accomplished via any one or more of motion recognition, image capture, thermal sensitivity, voice detection and so forth. In a simplest embodiment, the automatic entity 144 could simply be one or more cameras in proximity to the user 113, with associated image processing, software and such, identifying the user 112 through facial recognition, identifying skin and distinguishing marks including tattoos and markings, as a non-limited example. Or a multi-sensor device directed at the user 112 with more sensors, such as a microphone, speaker, thermal sensor, display, etc. In a more sophisticated embodiment, the automatic entity 144 can be an avatar or an AI persona using an AI engine to perform the validation/witnessing function. In a more sophisticated embodiment, the automatic entity 144 can be an avatar or an AI persona using an AI engine to perform the validation/witnessing function. For example, in one embodiment the AI persona is an electronic company representative, with delegated authority from a corporate business organization, acts on behalf and makes calculated decisions with authority granted by the corporate business organization. The AI persona is therefore working in lieu of a human actor, under delegated authority from a business organization, and resulting actions are audited and recorded into a verified witness record. This record indicates that intelligent decision making performed functions necessary to review, assess and witness said transactions. There is understood to be a widely flexible range of authority, from minute small decision making, ranging up to Executive Level decision making authority within the corporate entity constructs of business authority, finance and legal affairs decision conduct. In another embodiment, the AI persona is an electronic processing chip apparatus that performs reasonable sensory analysis, with flexible and programmable algorithms existing and that inventors anticipate extending and improving, where the resulting actions are audited and recorded into a verified witness record.

For flexibility and simplicity, the automatic entity 144 will be referred to using the generic term AI persona 144, understanding that in some embodiments, based on context and design objective, no AI engine need be used. However, in embodiments with an AI engine, the AI persona's 144 interactions with the user 112 may be varied and therefore less prone to deception (from the user 112). In some ways, the AI persona 144 can be a variant of the VSBC system 100, but not necessarily requiring interaction, acting instead as an observer of the user 112 to validate there is a real person in situ interacting with the VSBC system 100. For example, if the user 112 is purported to be a female, or of a certain height, such characteristics could be observed by the AI persona 144 and then correlated to the user-information in the VSBC system 100. Of course, the intent of the AI persona 144 is to recognize, using available sensor (s), that the witness 112 is a person and is the person interacting with the VSBC system 100 at the time of interaction. As is apparent, various and diverse validation or human-form recognition approaches are possible, only a few being provided above. Other approaches, too numerous to list, can and may be utilized depending on the sophistication desired by the implementing entity. As one demonstration of another non-limiting example, the AI persona 144 could request an eye-scan or other response (e.g., for identity matching or simply human identification) whereas the VSBC system 100 may not require the same from the user 112.

The AI persona 144 could be an VSBC originated observer or a non-VSBC originated observer. That is, as an VSBC originated observer, the health provider (or commercial company) would be responsible for the AI persona's witness role. For a non-VSBC originated observer, a third party, such as a federal agency or security corporation, for example, may be responsible for the AI persona's witness role. In this role the AI persona 144 can act as a digital notary, confirming that the user 112 is actually a person and the same person is performing the interactions with the VSBC system 100. Also, while the AI persona 144 as the role of a witness may validate the user's input information at the end of the user's interactions, it is possible for the AI persona 144 to also validate individual interactions as they are completed. For example, the VSBC system 100 may prompt the user 112 for a photograph, perhaps requesting a smile or a particular posture or position. The AI persona 144 could recognize the user's smiling motion or head posture or body position and use the timing of that data point to validate the “timing” of the photograph taken by the VSBC system 100. If desired, subsequent other actions could be monitored by the AI persona 144. Other forms could be the VSBC system 100 requesting the user 112 to place his/her hand into a fingerprint or biometric sensor. This motion of the user's hand could be registered by the AI persona 144 as being synchronous (in time relation) with VSBC system's 100 capture of the user's fingerprint. Various other approaches are only limited to the technology available and the level of sophistication desired by the implementing entity and as such are understood to be within the purview and scope of this disclosure.

In other embodiments, the AI persona 144 may be instituted via hardware co-situated with the VSBC system 100, such as for example, in a standalone kiosk, a service terminal 108, at a check-in location, and so forth. In other embodiments, the AI persona 144 may be a portable piece of hardware, for example a smart pad or smart phone, or other device with image and/or voice capturing capability. For the latter, a technician could bring such a device and direct it at the user 112 (or leave it with the user 112). Of course, any such AI persona 144 or digital witness would be in communication with the VSBC system 100 or a connected network 106. Upon verification, the AI persona 144 could send a verification stamp to the system or provide other means of verification, as desired.

To avoid an overriding of the AI persona 144 by a hacking source, various security controls can be utilized, including tokens tied to the session. The biometric witness 2-person application system, which can be described as a certified avatar, protects privacy and applies extended security for example, to social media events and real-time social media interaction. The captured and privatized biometric signatures when integrated with avatar-human interaction enforces anonymity, prevents non-authentic non-person robot bot access, and significantly reduces privacy breaches. Such integration of this system into electronic devices provides a unique and distinct capability. By ensuring the integrity, authenticity, and authorized usage of the persona actor, these security controls effectively prevent hacking and unauthorized manipulation.

For a practical non-limiting example in current technology, consider the following scenario: when a user initiates a session with the AI persona actor, the biometric identity markers are witnessed in real-time with proprietary methods disclosed, and then the system can generate a temporary session JWT token aka Communication Network activation ticket specific to the user. JWT stands for “JSON web token (JWT), which is an open standard (per Internet Engineering Task Force, Request for Comments 7519) that defines a compact and self-contained way for securely transmitting information between parties as a JSON object. Again, JWT is a standard, meaning that all JWTs are tokens, but not all tokens are JWTs.” This token facilitates the encapsulation of user information, encapsulates end-points and includes an expiration time. The temporary token is securely transmitted to the user and stored locally for verification purposes. A blockchain ledger can be utilized to record the creation and usage for session temporary JWT tokens. Each token creation and subsequent usage, such as authentication requests and persona actor interactions, are recorded as individual blocks in the blockchain. These blocks contain cryptographic hashes that guarantee the immutability and integrity of the recorded data. Additionally transparent and accessible audit records, resulting from the non-limited example above, can be stored into decentralized and transparent blockchain networks.

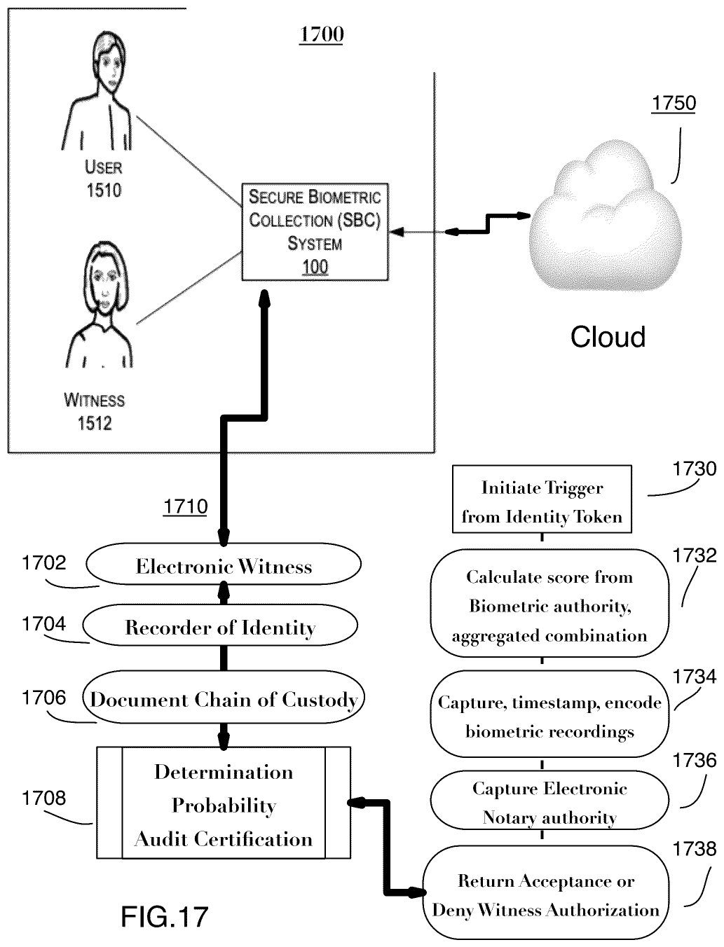

The use of an AI persona 144 or digital witness is understood to be applicable for the embodiment shown in FIG. 1 and as well for all other FIGs. having a witness (or confirming party) to the user's interactions and is not limited to health industry uses but may be applied to other industries desiring a human-equivalent witness or physical-equivalent notary or a service representative helping the user. An example of one possible electronic witness implementation is detailed in FIG. 17.

In all the above examples, the secure network 102 and VSBC system 100 may be powered by high-performance computers (HPCs) to carry out the transmission to an immutable ledger or blockchain step at a user's choice. However, if a user prefers to keep copies the secure transaction to be stored at a traditional storage unit or network due to cost concerns as the HPCs are very costly due to the sophisticated Graphic Processing Units (GPUs) and Central Processing Units (CPUs), the HPCs can be introduced at a later stage when cost is not an issue.

While FIG. 1 is characterized to be in the context of a health related transaction, it is well understood that the “verification” steps outlined above can be applied for use in any environment desiring authenticity of the user 112. For example, after verification the exemplary system may enable access to a social media site, limited access site, government, or for financial or high-value electronic or virtual transactions, and so forth.

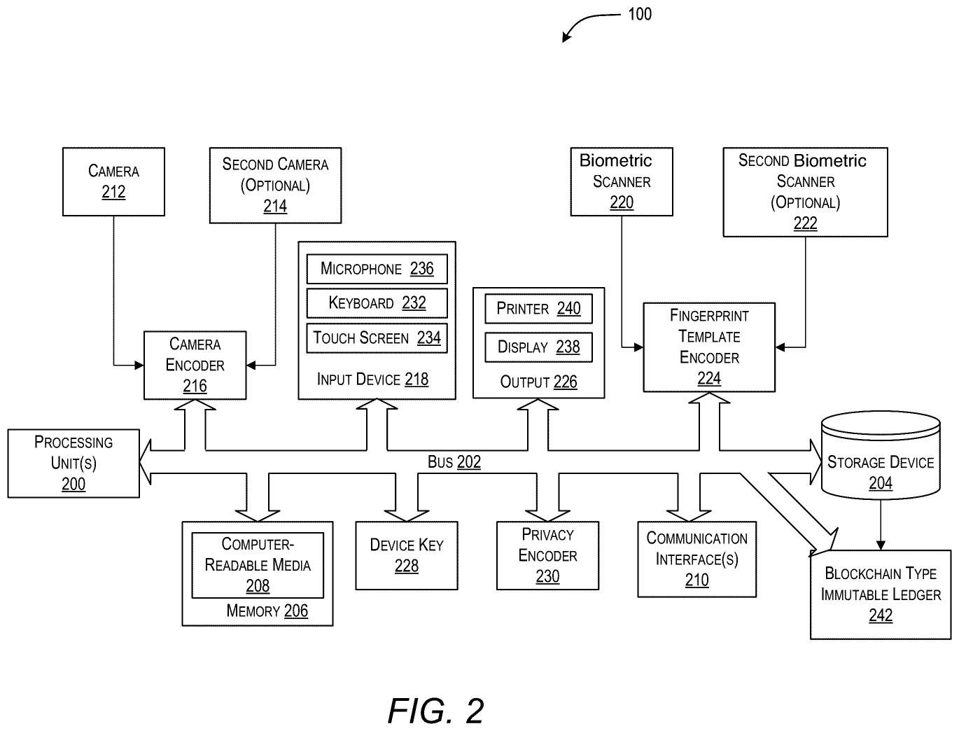

In FIG. 2, a system block diagram of an example of an implementation of the VSBC system 100 is shown in accordance with the present disclosure. The VSBC system 100 one or more processing units 200, a system bus 202, storage device 204, memory 206, computer-readable media 208, one or more communication interfaces 210, first camera 212, optional second camera 214, camera encoder 216, input device 218, first fingerprint (i.e., biometric) scanner 220, optional second fingerprint (i.e., biometric) scanner 222, fingerprint (i.e., biometric) template encoder 224, output device 226, device key 228, and privacy encoder 230. In this non-limiting example, the input device 218 may include keyboard 232, touch screen 234, and/or a microphone 236. The output device 226 may include a video display 238 and/or a printer 240.

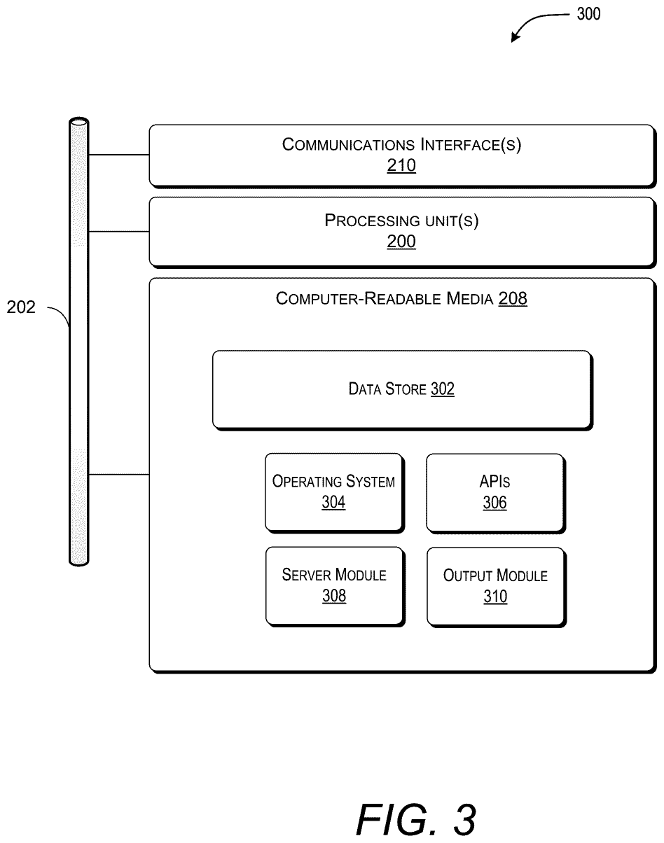

In this non-limiting example, the one or more processing units 200, storage device, memory 206, one or more communication interfaces 210, camera encoder 216, input device 218, output device 226, fingerprint (biometric) template encoder 224, device key 228, and privacy encoder 230 are in signal communication with system bus 202 and potentially each other. The camera encoder 216 is also in signal communication with the camera 212 and optional second camera 214 and the fingerprint (biometric) encoder 224 is also in signal communication with the fingerprint (biometric) scanner 220 and optional second fingerprint (biometric) scanner 222. In this non-limiting example, the one or more processing units 200 may be one or more processors as described in relation to FIG. 3. The storage device 204 may be a temporary memory device that stores information from the input device 218, one or more communication interfaces 210, camera encoder 216, fingerprint (biometric) template encoder 224, memory 206, one or more processing units 200, device key 228, and privacy encoder 230. The memory 206 may be a part of the storage device or storage that is separate from storage device. The memory 206 is configured to operate with the one or more processing units 200 and includes the 208 that stores computer-executable computer-readable media instructions that cause the one or more processing units 200 to perform the functions and methods of the VSBC system 100.

In this non-limiting example, the camera 212 may be a camera capable of capturing photographic images and/or video of the user 112 in the visible radiation spectrum. If the optional second camera 214 is present, the optional second camera 214 may be camera capable of capturing photographic images and/or video of the user 112 in the invisible radiation spectrum including ultraviolet spectrum radiation, near-infrared spectrum radiation, or far-infrared spectrum radiation.

Moreover, in this non-limiting example the fingerprint (biometric) scanner 220 is located on the VSBC system 100 and is configured to scan one or more fingerprints (biometrics) of the user 112. The fingerprint (biometric) scanner 220 may be a signal scanner that is configured to scan the fingerprint (biometric) of a single finger of the user 112 or one or more fingerprint (biometric) scanner that are configured to scan multiple fingers of the user 112. In this non-limiting example, the user 112 and witness 114 (or AI persona 144) may utilize a single fingerprint (biometric) scanner 220 to scan the fingerprints (biometrics) of the both the user 112 and witness 114 (acting as a second person as described previously, or verifying AI persona 144). Alternatively, the VSBC system 100 may include the optional second fingerprint (biometric) scanner 222 to scan the fingerprint of the second person (such as the witness 114 or proximately located service representative 110). If the optional second fingerprint (biometric) scanner 222 is configured to scan the fingerprint of a second person that is either the witness 114 or a proximately located service representative 110, the optional second fingerprint (biometric) scanner 222 may be part of the VSBC system 100. If, instead, the optional second fingerprint (biometric) scanner 222 is configured to scan the fingerprint of a second person that is a remotely located service representative 110 (e.g., the service representative 110 may be located in another area of the same building, another town or city, another state, or even another country), optional second fingerprint (biometric) scanner 222 would be located remotely at the service terminal 108 of the service representative 110. In this non-limiting example, the fingerprint (biometric) of the service representative would be scanned at the optional second fingerprint (biometric) scanner 222 and transmitted to the fingerprint (biometric) template encoder 224 via the service terminal 108, secure server 104, and network 106.

Furthermore, in this non-limiting example, the input information from the user 112, photographic/video of the user 112, scanned fingerprints of the user 112 and/or witness 114 or service representative 110 is initially unprotected data. The camera encoder 216 and fingerprint (biometric) template encoder 224 are hardware components and/or software modules that are configured to encode this unprotected data into encoded data that is then stored in the storage device 204. This encoded data may then be further encoded by the privacy encoder 230 utilizing the device key 228. The device key 228 is a private encryption key that is only known to the VSBC system 100 and may be implemented, for example, by a privacy encoder chip. The VSBC system 100 can also be designed with HPCs that will enable the transactions and its encryption keys being recorded on an immutable ledger such as a blockchain instead of, or in combination with, storage device 242.

It is appreciated by those skilled in the art that the circuits, components, modules, and/or devices of, or associated with, the VSBC system 100 are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical, such as, for example, conductive wires, electromagnetic wave guides, cables, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying digital formats without passing through a direct electromagnetic connection.

Turn to FIG. 3, a system block diagram of an example of an implementation of components 300 of the VSBC system 100 is shown in accordance with the present disclosure. The components 300 are configured to enable the VSBC system 100 to securely collect and record data from the user 112 associated with a secure transaction. The components 300 may represent one or more of the devices shown in FIG. 2 that include, for example, the one or more processing units 200, system bus 202, memory 206, computer-readable media 208, one or more communication interfaces 210, camera encoder 216, fingerprint (biometric) template encoder 224, device key 228, and privacy encoder 230. The components 300 are in signal communication and operatively connected, for example, via the system bus 202, which may include one or more of a system bus, a data bus, an address bus, a PCI bus, a Mini-PCI bus, and any variety of local, peripheral, and/or independent buses.

As utilized herein, processing unit (s), such as the processing unit (s) 200 may represent, for example, a CPU-type processing unit, a GPU-type processing unit, a field-programmable gate array (“FPGA”), another class of digital signal processor (“DSP”), or other hardware logic components that may, in some instances, be driven by a CPU. For example, and without limitation, illustrative types of hardware logic components that may be utilized include Application-Specific Integrated Circuits (“ASICs”), Application-Specific Standard Products (“ASSPs”), System-on-a-Chip Systems (“SOCs”), Complex Programmable Logic Devices (“CPLDs”), etc.

As utilized herein, computer-readable media, such as computer-readable media 208, may store instructions executable by the processing unit (s) 200. The computer-readable media may also store instructions executable by external processing units such as by an external CPU, an external GPU, and/or executable by an external accelerator, such as an FPGA type accelerator, a DSP type accelerator, or any other internal or external accelerator. In various examples, at least one CPU, GPU, and/or accelerator is incorporated in a computing device, while in some examples one or more of a CPU, GPU, and/or accelerator is external to a computing device.

Computer-readable media may include computer storage media and/or communication media. Computer storage media may include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random-access memory (“RAM”), static random-access memory (“SRAM”), dynamic random-access memory (“DRAM”), phase change memory (“PCM”), memory read-only (“ROM”), erasable programmable read-only memory (“EPROM”), electrically erasable programmable read-only memory (“EEPROM”), flash memory, compact disc read-only memory (“CD-ROM”), digital versatile disks (“DVDs”), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

The one or more communication interfaces 210 may represent, for example, network interface controllers (“NICs”) or other types of transceiver devices to send and receive communications over a network.

In the illustrated example, computer-readable media 208 includes the data store 302. In some examples, the data store 302 includes data storage such as a database (including a blockchain structure), data warehouse, or other type of structured or unstructured data storage. In some examples, the data store 302 includes a corpus and/or a relational database with one or more tables, indices, stored procedures, and so forth to enable data access including one or more of hypertext markup language (“HTML”) tables, resource description framework (“RDF”) tables, web ontology language (“OWL”) tables, and/or extensible markup language (“XML”) tables, for example.

The data store 302 may store data for the operations of processes, applications, components, and/or modules stored in computer-readable media 208 and/or executed by one or more processing units 200 and/or accelerator (s).

In this non-limiting example, the computer-readable media 208 also includes operating system 304 and application programming interface (s) (APIs) 306 configured to expose the functionality and the data of the components 300 to external devices associated with the VSBC System 100. Additionally, the computer-readable media 204 includes one or more modules such as the server module 308 and an output module 310, although the number of illustrated modules is just an example, and the number may vary higher or lower. That is, functionality described herein in association with the illustrated modules may be performed by a fewer number of modules or a larger number of modules on one device or spread across multiple devices.

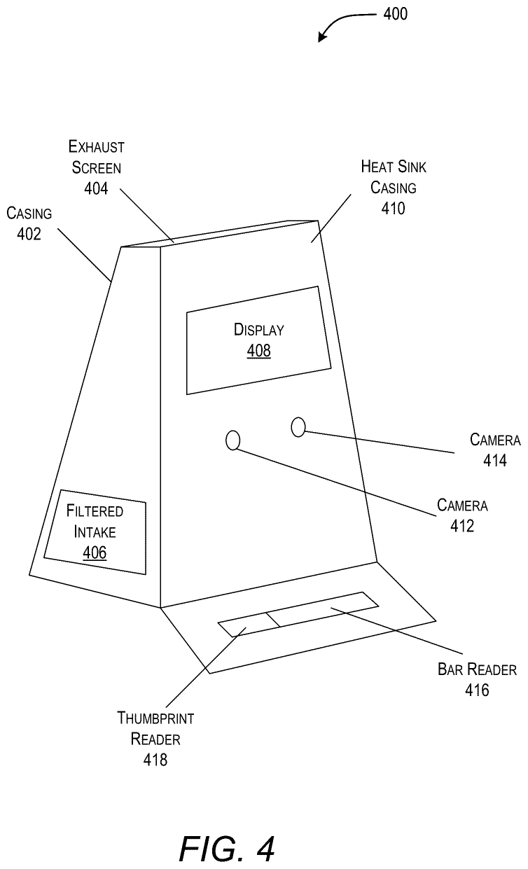

FIG. 4, a perspective front view of an example of an implementation of the VSBC system 400 is shown in accordance with the present disclosure. In this non-limiting example, the VSBC system 400 includes a casing 402 (i.e., a housing), an exhaust screen or vents 404, a filtered intake 406 to remove particulate matter and improve reliability of the device in hot and/or dusty field conditions, a display 408 to display instructions for a user 112, a heat-sink casing 410 (which, for example, may be constructed of metal) to dissipate camera heat or the like, a first camera 412 and an optional second camera 414, where the first camera 412 may be configured to sense normal visible spectrum light and the optional second camera 414 may be configured to sense thermal, infrared or non-visible spectrum light, for example.

The VSBC system 400 further includes a fingerprinting portion including, for example, a bar reader 416 configured for multiple-finger scanning and a thumbprint reader 418 configured for thumb or single-finger scanning. The VSBC system 400 alternatively include a first fingerprint scanner (not shown but described in FIG. 2) and an optional second fingerprint scanner (not shown but described in FIG. 2).

In this non-limiting example, casing module 402 of the VSBC system 400 may have generally trapezoidal shape in its side view and substantially rectangular shape in its front view. It is appreciated to those of ordinary skill in the art that these shapes are for example purpose only and the casing module 402 may vary significantly based on its design and intended use. The exhaust screen or vents 404 may be formed towards the top and/or side parts of the VSBC system 400, through which heated or exhaust air flows outwards. The intake vent filter 406 may be formed towards the bottom part of the casing module 402, through which ambient air flows inwards.

The display screen 408 may be positioned and located on the front face of the VSBC system 400 with a generally rectangular shape. The first camera 412 and optional second camera 414 may be position below the display screen 408 where the two cameras 412 and 414 are aligned in the horizontal direction. In this non-limiting example, the first camera 412 is for visible light and the second camera 414 is for non-visible light, such as infrared or ultraviolet light, but not limited thereto.

The heat-sink casing 410 may be constructed of high thermal conductivity material to dissipate the heat generated by the cameras 412 and 414 and other components of the VSBC system 400. In this non-limiting example, the heat-sink casing 410 may be constructed of metal. The fingerprint collecting module may be positioned at the bottom part of the VSBC system 400. The fingerprint collecting module may include at least two fingerprint readers, a first fingerprint reader 416 and a second fingerprint reader 418 that are located adjacent to each other. Among the two fingerprint readers, one fingerprint reader may have an elongated shape that is configured for multiple-finger scanning, while the other fingerprint reader may be configured for single-finger scanning, such as, for example, thumb scanning.

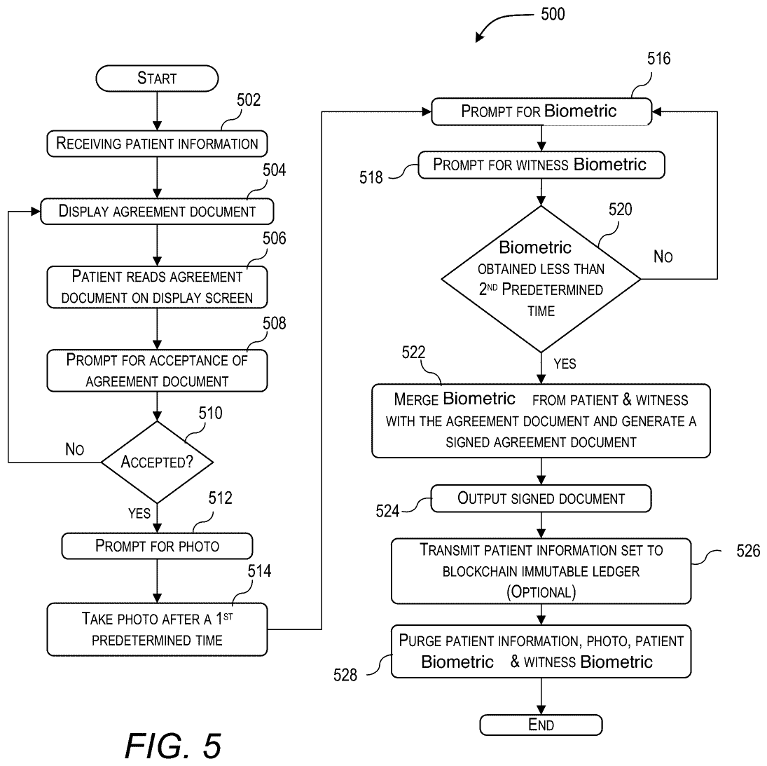

As an example of operation, FIG. 5 is a flowchart diagram for an example of an implementation of a collecting method 500 performed by the VSBC system 100 in accordance with the present disclosure. The method 500 begins 502 by the VSBC system 100 receiving a user's 112 personal information. In this non-limiting example, the method 500 is related to a medical provider and the user 112 is a patient that is attempting to receive medical services from the medical provider. The VSBC system 100 then produces and displays 504 an agreement document on the display 238 for the user 112 to accept and digitally sign via biometric inputs. The user 112 then reads the agreement document that is displayed on the display 238. The VSBC system 100 then prompts 508 the user 112 for acceptance of the terms of the agreement document. In decision step 510, if the user 112 does not accept the agreement document, the method returns to step 504 and the agreement document is again displayed on the display 238.

If, instead, the user 112 does accept the agreement document, the VSBC system 100 prompts 512 the user 112 for a photograph and/or video recording. The user 112 may be informed to not move for a first predetermined time and at the expiration of the first predetermined time, the VSBC system 100 takes 514 a photograph and/or records video of the user 112 with at least the first camera 212. In this non-limiting example, the first predetermined time may be, for example, between approximately three to five seconds. The VSBC system 100 then prompts 516 the user 112 for a fingerprint (biometric) and after the user 112 provides a fingerprint (biometric) scan with the fingerprint (biometric) scanner 220 the VSBC system 100 prompts the witness 114 or service representative 110 for a fingerprint (biometric) within a second predetermined time. The witness 114 or service representative 110 then provide a fingerprint (biometric) scan with either the fingerprint (biometric) scanner 220 or optional second fingerprint (biometric) scanner 222. If an AI persona 144 is utilized, the AI person 144 would replace the role of the witness 114 and provide the appropriate responses or confirmation action to the VSBC system 100. In this non-limiting example, the VSBC system 100 may first timestamp the time when the fingerprint (biometric) scan of the user 112 is received and again second timestamp the time when the fingerprint (biometric) scan of the second person is received. Moreover, in this non-limiting example, the second predetermined time may be, for example, approximately 1000 milliseconds. The VSBC system 100 then determines if the second fingerprint (biometric) scan provided by either the witness 114 (or AI persona 144) or service representative 110 was obtained within the second predetermined time by comparing the first and second timestamps. In decision step 520, if the VSBC system 100 determines that the second fingerprint (biometric) scan was obtained after the second predetermined time, the method then returns to step 516 and the VSBC system 100 again prompts 516 the user 112 and prompts 518 the second person for their fingerprints (biometrics). The VSBC system 100 again determines if the second fingerprint (biometric) scan was obtained after the second predetermined time. If yes, the sub portion of the method between steps 515 to 520 repeats.

If, instead, the VSBC system 100 determines that the second fingerprint (biometric) scan was obtained within the second predetermined time after the fingerprint (biometric) scan of the user 112 was obtained, the method continues to step 522. In step 522, the VSBC system 100 merges the fingerprint (biometric) scans from both the user 112 and the second person (i.e., either the witness 114 or AI persona 144 or service representative 110) with the agreement document and generates a signed agreement document. The VSBC system 100 then outputs a copy of the biometrically “signed” agreement document with the display 238 and/or printer 240. Upon user's 112 instruction, the VSBC system 100 can optionally transmit 526 the user's 112 information, photograph/video, scanned fingerprint (biometric), and the scanned fingerprint (biometric) of the second person to a secured blockchain or immutable ledger or alternatively purges 528 these data without transmitting them to a secured blockchain or immutable ledger. The method 500 then ends.

In an example of operation, the VSBC system 100 may record transactions in real-time such as may be required for auditing purposes. In general, the present disclosure relates to a collecting apparatus, such as segregated custom-purpose hardware but not limited thereto, that reads, encodes and records a user's 112 biometric data, and purges all raw and intermediate biometric data once finalized or encrypted data has been provided for secure storage or an immutable ledger like a blockchain. The VSBC system 100 may delete the original biometric data upon delivery of “signed” agreement document to the storage device 204. The “signed” agreement document and the resulting biometric data in an encoded and encrypted data set may be transmitted to remote secure storage or an immutable ledger at the secure server 104 for permanent storage. In this non-limiting example, the VSBC system 100 may encrypt (i.e., scramble and encode) the private information in a manner that cannot be reasonably deciphered outside of the VSBC system 100. This may allow for permanent storage at the secure server of such biometric information, such as scrambled without external cipher keys but not limited thereto, with reduced risk of compromise by, for example, code viruses, theft and/or loss of system data from cloud, private network, or insurance industry data warehouse systems.

While various embodiments have been shown for descriptive purposes, it shall be understood by those of ordinary skill in the art that such and like embodiments may be adapted to complex systematic and biometric workflow systems, for example. Moreover, there are numerous possibilities and variations in the medical, finance and other marketplaces to which embodiments of the present disclosure may be directly applied or reasonably adapted.

For example, the VSBC system 100 may include the first camera 212 and the optional second camera 214. In this non-limiting example, one of the two cameras 212 or 214 may be for visible light and the other for non-visible light, and the two cameras 212 and 214 may be aligned to take a photograph and/or record video in the same direction. Furthermore, the fingerprint (biometric) scanner 220 may include two fingerprint (biometric) readers, where one of the two fingerprint (biometric) readers is for one finger (biometric), such as a thumbprint reader, and the other fingerprint (biometric) reader is for multiple fingers (biometrics) such as a fingerprint bar reader. Moreover, the two fingerprint (biometric) readers may be located proximate to each other so that two or more immediate or substantially simultaneous fingerprints (biometrics) can be generated from the plurality of fingerprint (biometric) readers. Thus, some form of time-limited response metric is determined, further verifying proximity.

In this disclosure, the collecting methods described may be applied to private information including, for example in a medical provider example, the name of patient/user 112, identifying number of patient, and/or date of birth of patient, where the response includes an affirmative response or a negative response. The collecting method may be applied where the certain time period is, for example, 10 seconds. The collecting method may be applied where the two persons include, for example, the patient/user 112 and a witness 114 or service representative 110. As noted above, the exemplary system can be implemented as a secure form of user validation to allow connection or access to a social media site, restricted access site, government, or for financial or high-value electronic or virtual transactions, and so forth.

Using the VSBC system 100 and example method together as described herein, patient privacy may be protected while recording highly private personal data about each individual. This is a challenging application, particularly considering that high-level government and insurance industry regulations already require, in a systematic way, collection of biometric information such as fingerprints, photographic images, and other data based upon personal characteristics of individuals.

In this disclosure, the VSBC system 100, or parts thereof, may be implemented in dedicated hardware and/or software without limitation. For example, while a highly-specific, specialized apparatus is provided to protect patient privacy while recording private and personal data about an individual, at least some of the functionality may optionally be embodied in software. Such solutions are adaptable based on specific requirements, such as, for example, government and/or industry regulations or goals, where collecting biometric information is either an end-goal or but a small step in a larger process. For example, positive identification readers may improve safety and facilitate audits of potential financial fraud abuses in complex high-value industries. Along with the adoption of blockchain structure, any new data or fraud abuses may be identified instantaneously by the user or the custodian of the data located on the distributed blockchain network.

It is appreciated by those of ordinary skill in the art that embodiments of the present disclosure may incorporate existing fingerprint reader devices, biometric lock devices, access-authorization-auditing electronic system access controls, and healthcare data processing systems and databases. For example, embodiments may be adapted for computing devices with integrated fingerprint readers, fingerprint reader hardware in law enforcement and customs identification applications, biometric door locks, systematic face scanning and/or facial recognition, financial industry transaction systems, security agency hardware encryption, or the like.

As an example, turning back to FIG. 4, the VSBC system 400 may be disposed in a sealed enclosure, such as for intermittent use and/or with an external heat-sink. As described earlier, the VSBC system 100 may include a casing module, a display screen 408 which displays written instructions provided to the patient, a metal heat-sink casing for dissipating camera heat, one or two cameras 412 or 414 for acquiring images based on normal visible light as well as thermal, infrared or non-visible-light, a fingerprint bar reader 416 for multiple-finger scanning; and a thumbprint reader 418 for thumb or single-finger scanning.

The VSBC system 400 may have a trapezoidal shape in its side view and a substantially rectangular shape in its front view, or may be shaped differently to increase surface area for heat dissipation. The display screen 408 is preferably provided in the front face of the VSBC system 400 and may have a rectangular shape. The camera 412 is provided below the display screen 408, including in the two different cameras 412 and 2414 aligned in the height or vertical direction, where one of the two cameras 412 or 414 is for visible light and the other is for non-visible light. The casing 402 is made of material that readily dissipates heat generated by the camera 412 and 414, and is preferably made of metal.

In this non-limiting example, fingerprint (biometric) scanner is located at a bottom part of the VSBC system 400, including at least one of the two fingerprint (biometric) readers 416 and 418, which are located adjacent to each other. Among the two fingerprint (biometric) readers 416 and 418, one fingerprint (biometric) reader (i.e., bar reader 416) which may have a more elongated shape may be used for multiple-finger scanning, while the other fingerprint (biometric) reader (i.e., thumbprint reader 418) may be used for single-finger scanning, such as, for example, thumb scanning. A sensor data purging module is provided inside the casing 402, and configured to purge all temporary data acquired by the photo collecting module and the fingerprint collecting module.

In general, the VSBC system 400 includes acquisition hardware and other hardware mechanisms such as at least one encoding and/or recoding device, which operate in unison where raw biometric data is input. This unprotected data is encoded by hardware and/or software before delivery to temporary storage (i.e., storage device 204) based on an encryption chip mechanism. The VSBC system 400 utilizes the private encryption key 228 which is known only to the VSBC system 400. The hardware encryption apparatus, and the hardware encryption mechanism, may comprise a uniquely coded privacy encoder chip.

In this non-limiting example, the method performed by the VSBC system 400 demonstrates how a private biometric signature may be used to confirm a real-time medical transaction, for example. For the prevention of medical fraud, the method steps demonstrate how a patient/user 112 may review a document and then certify with a witness 114 (or AI persona 144), using a dedicated real-time apparatus with hardware encryption in this non-limiting example. Further, the biometric information of both the patient/user and the witness may be recorded on the immutable ledger such as a blockchain. In the event of a fraud occurs, the fraudulent transaction can be immediately pin-pointed by locating the new block of data that contains fraudulent actor's information.

Similar to the previous discussion, the VSBC system 400 performs a method that includes receiving patient information, generating an agreement document that is displayed on the display 408 for the patient to read. Then, the VSBC system 400 reads, records and encodes, prompts for acceptance or non-acceptance. If the patient matching the biometric data acknowledges and accepts the document, a corresponding medical identification number is generated or input, and each of the patient's surname, given name, and date of birth are input or confirmed. Next, the VSBC system 400 displays a countdown timer with optional instructions for a photograph, and at least one visible-light camera acquires a photographic image, which it saves to temporary storage (i.e., the storage device 204). Similarly, the non-visible-light camera acquires an image and saves that in temporary storage as well. The photographic encoder chip combines and/or hashes the two photographic images into a combined data set, and forwards the combined data set, including the encoded private photographic images, to a privacy encoder temporary storage area on the storage device 204.

The VSBC system 400 also displays instructions for fingerprint (biometric) capture, and actuates at least one fingerprint (biometric) scan. It processes the fingerprint (biometric) scan into a fingerprint (biometric) template data set; and a fingerprint (biometric) encoder chip forwards a combined data set including unencrypted fingerprint (biometric) template data to the privacy encoder temporary storage. The privacy encoder communicates with a device key chip delivering an encryption key unique to session recording, and then combines and independently encrypts all data acquired including agreement, patient name, date of birth, patient responses, encoded photographic image data, and encoded fingerprint template data into an encrypted data set. The VSBC system 400 purges all temporary data including unencrypted photographic images, fingerprint (biometric) scans, and fingerprint (biometric) templates such that the combined mechanisms delete original biometric data within the VSBC system 400 upon delivery to the secure server 104 that could be embedded with a blockchain structure and/or software. Thus, the VSBC system 400 delivers the resulting biometric data in an encoded and/or encrypted data set to the secure server 104 for permanent storage.

In general, the VSBC system 400 is a hardware encryption device that is preferably embodied in a compact, durable form including a camera module having a first camera for acquiring visible-light images and a second camera for acquiring non-visible-light images, such as infrared and/or ultraviolet, where the two cameras align to acquire images from substantially the same direction. The device includes a fingerprint (biometric) module including a first fingerprint (biometric) reader preferably for one finger, and a second fingerprint (biometric) reader preferably for multiple fingers, where the second fingerprint (biometric) reader is preferably located proximate to the first fingerprint (biometric) reader. A privacy module includes a first chip that converts acquired photographic images into an encoded photo data set, and a second chip that converts acquired fingerprints into an encoded fingerprint (biometric) data set template. A device key chip is connected to the privacy module for providing a unique encoded symmetric device key. A hardware encryption processor (which may be one of the one or more processing units 200 or a separate processor) is connected to the privacy module. All modules are physically separated with hardware connection boundaries that precludes malicious virus software, or the like, such that only the hardware encryption processor is connected to any computing main board.

In this non-limiting example, the recording method is embodied within a compact device for completing a medical transaction declaration record, such that a real-time transaction encodes in a manner preventing forgery or tampering. The method includes activating at least two fingerprint (biometric) readers that, when activated, requires two immediate and substantially simultaneous fingerprints (biometrics). The device display prompts for camera photographic images, and then acquires photographic images with the cameras. The VSBC system includes a hardware encryption module for encoding and encrypting of the recorded record data.

When the VSBC system prompts for photographic images, the patient may wait a first predetermined time (such as, for example, 3-5 seconds) for the photographic images to be acquired; and, if the patient does not accept the document, the process returns back to the first step. After taking the photographic images successfully, the process goes on to the next step, where biometric fingerprints are required to confirm the identities of the patient and the witness (or AI persona). The fingerprints (verification) from these two “persons” are recorded on separate physical reader devices. Next, the VSBC system time-stamps each biometric fingerprint and electronically determines that the fingerprints were recorded within a certain time period defined by the second predetermined time that may be, for example, about one to 10 seconds.

The VSBC system produces an “agreement document”, such as using Portable Document Format (PDF), and stores it in device memory. It displays the “agreement document” on the device display 408. The VSBC system then records an affirmative or negative response from the patient through the device display 408 and at least one user-selectable response. It prompts for biometric reader activation, and records biometric fingerprints from two persons, on, for example, two physical reader devices. The VSBC system time-stamps each biometric fingerprint and electronically determines that that fingerprints were both recorded within the second predetermined time that may be, for example, between about 1 second to 10 seconds of each other.

The VSBC system is configured to make an electronic decision about the physical proximity of the two people (i.e., how physically close are they to each other), such as one patient/user 112 and one witness 114 (or AI persona 144), based upon the first fingerprint (biometric) reader time-stamp and the second fingerprint (biometric) reader time-stamp (or if the AI persona 144 is used, a verification signal). If the VSBC system determines that the fingerprints (biometrics/verifications) were recorded within a predetermined period, such as 10 seconds or less, the VSBC system merges the biometric signatures from the two persons with the “agreement document,” thereby generating the “signed agreement document.” However, if VSBC system determines that the fingerprints (biometrics/verifications) were not recorded within 10 seconds of each other, the process returns to the fingerprint (biometric) scan step and the process repeats. When the VSBC system has merged the biometric signature in the signed agreement document, the signed agreement document is output to the user 112 and sent to the secure server 104 and the process is complete.

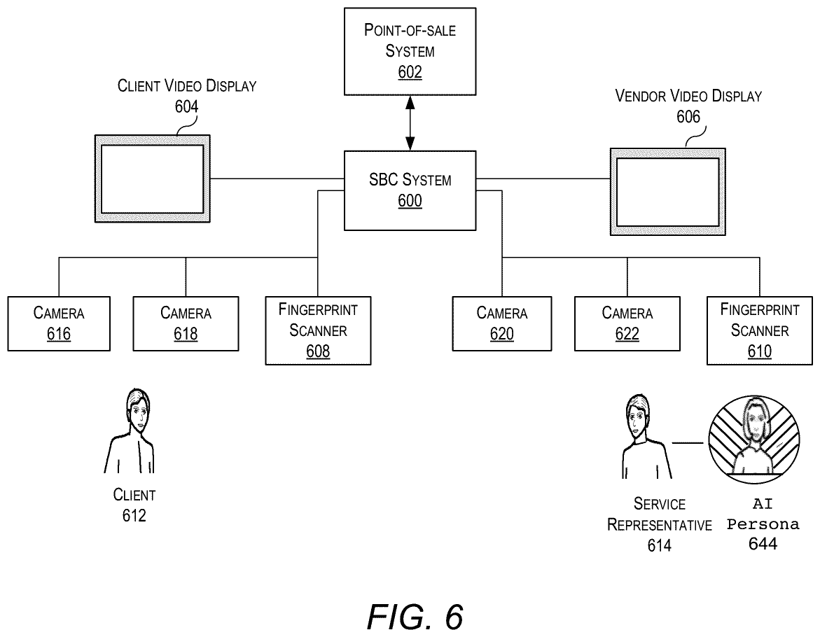

FIG. 6 is a system block diagram of an example of an implementation of the VSBC 600 for a point-of-sale application on a point-of-sale system 602 for purchasing various materials or non-fungible transactions in accordance with the present disclosure. In this non-limiting example, the VSBC 600 is in signal communication with the point-of-sale system 602 and a client video display 604 and a vendor video display 606. The client video display 604 is a display monitor having a 180-degree viewing range and the vendor video display 606 is another display monitor also having a 180-degree viewing range. In this non-limiting example, the client video monitor 604 and vendor video display 606 may be oriented such that both monitors are facing opposite to each other. In general, the client video display 604 and vendor video display 604 are configured to simultaneously display the same identical transactional data. This may be accomplished via specialized hardware circuitry, software, or both within the VSBC system 600. In general, the VSBC system 600 controls both the outputs to the client video display 604 and vendor video display such that both monitors display mirrored content from the VSBC system 600 and point-of-sale system 602. In this non-limiting example, the VSBC system 600 may include biometric capture readers such as, for example, the first fingerprint scanner 608 and second fingerprint scanner 610. In this non-limiting example, the first fingerprint scanner 608 may be utilized by a client 612 (i.e., a user) that desires to purchase the material or enter into various non-fungible transactions with a vendor. The second fingerprint scanner 610 may be utilized by a service representative 614 of the vendor. Similarly, the client video display 604 is configured to be utilized by the client 612 and the vendor video display 606 is configured to be utilized by the service representative 614. In addition to the fingerprint scanners 608 and 610, the VSBC system 600 may be in signal communication with a first visual spectrum camera 616, a first non-visual spectrum camera 618, a second visual spectrum camera 620, and a first non-visual spectrum camera 622, respectively. In this non-limiting example, the first visual spectrum camera 616 and the first non-visual spectrum camera 618 may be configured to record photographs and/or video of the client 612 and the second visual spectrum camera 620 and the second non-visual spectrum camera 622 may be configured to record photographs and/or video of the service representative 614. Moreover, while not shown, it is appreciated that the VSBC system 600 may further include one or more microphones to record the voices of the client 612, service representative 614, or both.

As discussed in the embodiment of FIG. 1, a digital witness or AI persona 144 can be instituted wherein the service representative 614 is replaced with the AI persona 144. And appropriate point-of-sale management is proctored by the AI representative.

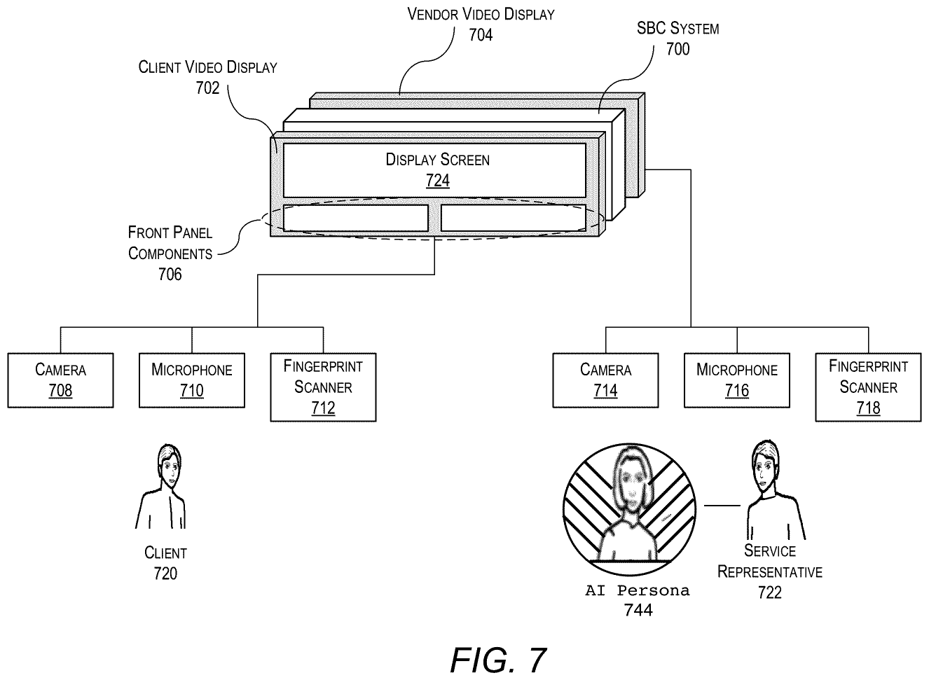

FIG. 7 is a system block diagram of an example of an implementation of the VSBC system 700 in a compact, audio-capture recorder form in accordance with the present disclosure. In this non-limiting example, the VSBC system 700 and may be in signal communication with or include a point-of-sale system. The VSBC system 700 is in signal communication a client video display 702 and vendor video display 704. In this non-limiting example, the client video display 702 includes front panel components 706 and the vendor video display 704 also includes back panel components (not shown). The front panel components 706 includes a camera 708, microphone 710, and fingerprint (biometric) scanner 712. Similarly, the back panel components include a camera 714, microphone 716, and fingerprint (biometric) scanner 718. The front panel components 706 are configured to be utilized by a client 720 and the back panel components are configured to be utilized by a service representative 722. In this non-limiting example, the VSBC system 700 may optionally not include the camera 708 and camera 714 and, instead, capture voice or audio recordings in addition to hand and fingerprints with the microphone 710 and 716 and fingerprint (biometric) scanners 712 and 718. Such variation provides simplicity in design and function. In this non-limiting example, the combination of VSBC system 700 and client video display 702 includes a front panel including a display screen 724 and the front panel components 706 includes human interface readers, specifically at-least microphone 710 and biometric hardware reader device such as, for example, the camera 708 and the fingerprint scanner 712. In this non-limiting example, the combination of VSBC system 700 and vendor video display 704 includes a back panel including a display screen (not shown) and the back panel components includes human interface readers, specifically at-least microphone 714 and biometric hardware reader device such as, for example, the camera 716 and the fingerprint scanner 718. Similar to the last example, the client video display 702 and vendor video display 704 are oriented opposite each other. In this non-limiting example, the VSBC system 700, and client video display 702, and vendor video display 704 may include a waterproof sealant membrane designed for application of chlorides or alcohol based cleaner solutions without damaging internal electronics. As in FIG. 1 and FIG. 6, an AI persona 744 may be implemented, according to design preference.

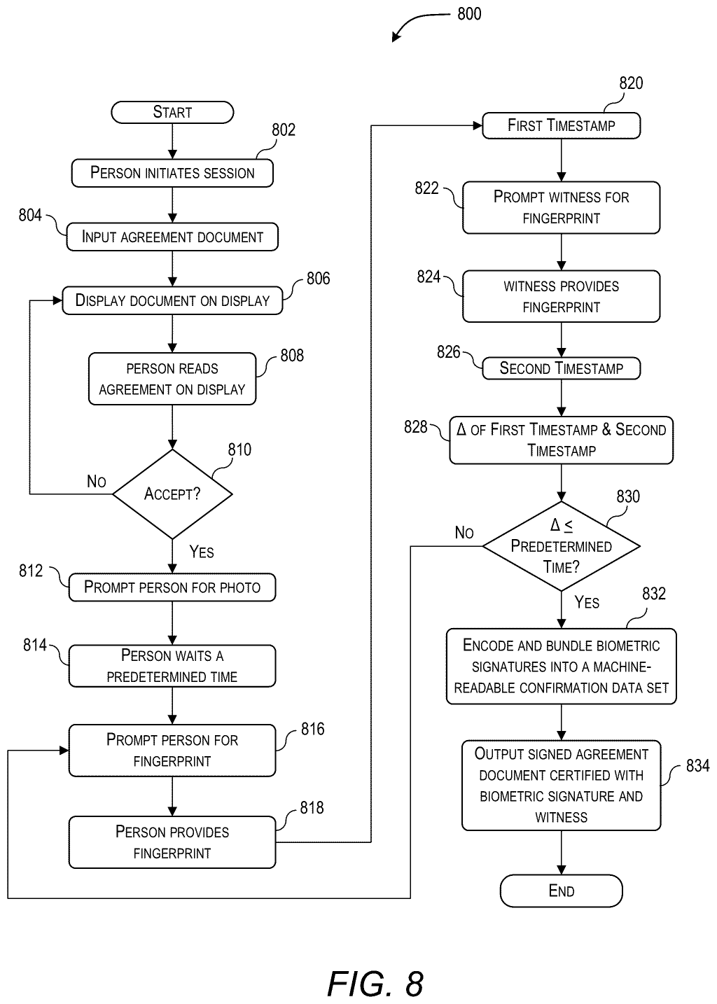

FIG. 8 is a flowchart diagram for an example of an implementation of a method 800 for encoding private biometric data into an audit record in accordance with the present disclosure. The method 800 starts by the user 112 initialing 802 a session on the VSBC system. The VSBC system queries the user 112 for their information in generating an agreement document and the user 112 inputs 804 the requested information. Once the information from the user 112 has been entered, the VSBC system displays 806 (for example, and a screen of a display system of the VSBC system) the completed agreement document for the user 112 to review. The user 112 then reads 808 the agreement document on the display and, in decision step 810, the VSBC system queries the user 112 to accept the agreement document. If the user 112 does not accept the agreement document, the VSBC system returns to step 806 and again displays the agreement document for the user to accept and then again queries the user 112 to accept the agreement document. If the user 112 continues to not accept the agreement document, the VSBC system may optionally end the session.

If, instead, the user 112 accepts the agreement document, the VSBC system then prompts 812 the user 112 that a photograph and/or video recording will be obtained with the camera after a predetermined time that may be, for example, 3-5 seconds and then then the user waits 814 the predetermined time and the VSBC system takes a picture or video of the user 112. The VSBC system then prompts 816 the user 112 for a fingerprint (biometric) and the user 112 then provides a fingerprint (biometric) with the fingerprint (biometric) scanner. Once the VSBC system receives the fingerprint (biometric) from the user 112, the VSBC system timestamps 820 the scanned fingerprint (biometric) with a first timestamp. The VSBC system then prompts 822 a second person for a fingerprint (biometric) and the second person then provides 824 their scanned fingerprint (biometric). If the second person is the AI persona (not shown), a verification of the user 112 is sent. Once the VSBC system receives the fingerprint (or verification from AI persona) from the second person, the VSBC system timestamps 826 the scanned fingerprint (biometrics/verifications) with a second timestamp. The VSBC system then calculates 828 the difference in time between the first timestamp and the second timestamp and then determines if the difference in time is less than or equal to a second predetermined time which may be, for example, about 1,000 milliseconds. If the difference in time is greater than the second predetermined time, the method 800 returns to step 816 and the VSBC system again prompts the user 112 for a fingerprint (biometric) and the method 800 repeats until decision step 830. If, instead, the difference is less than or equal to the second predetermined time, the VSBC system encodes 832 and bundles the biometric signatures into a machine-readable confirmation data set that may be stored in storage device 204 or transmitted to a blockchain type immutable ledger. The signed agreement that is certified with biometric signatures of the user 112 and the second person is output 834 for the user 112 to review. The VSBC system may then purge all of the biometric data and the method 800 ends. In this non-limiting example, as described earlier, the second person may be a witness 114 or AI persona 144 or customer service representative 110.

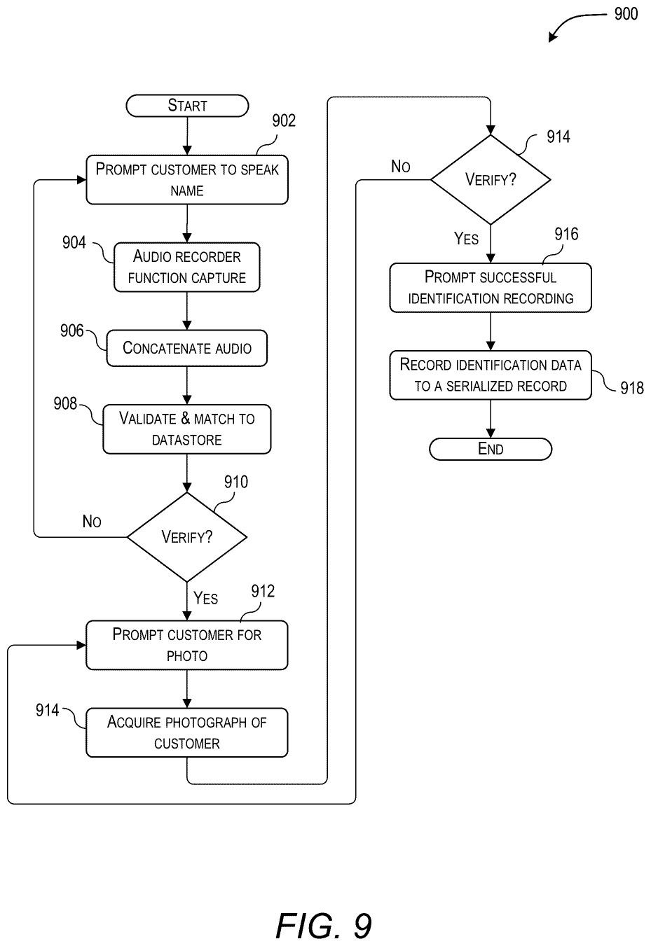

FIG. 9 is a flowchart diagram of an example of an implementation of a method 900 for audio-capture recorder logic applied in compact the VSBC system 700 in accordance with the present disclosure. The method 900 starts by prompting 902 the customer (i.e., user 112/client 720) to speak their name with either video or audio instructions on the display screen 724 or over a speaker. The VSBC system 700 then records 904 the customers voice with an audio recorder function capture and then performs 906 a quality, compaction, and noise removal via an electronic process generally referred to a concatenation. The VSBC system 700 then validates 908 and matches the recorded audio to data in a database or private store and, in decision step 910, if the recorded audio is not validated, the method 900 returns to step 902 and the process repeats to decision step 910 because the VSBC system 700 will either cancel the transaction or re-attempt to process the current transaction recording process.

If instead, the recorded audio is validated, the method 900 prompts 912 the customer for a photo and then acquires 914 a photograph of the customer utilizing a timer mechanism. In decision step 914, the VSBC system 700 compares the acquired photograph to a private image database resulting in continuation, cancellation, or re-attempt of the existing transaction. If the verification fails, the process returns to step 912 and the VSBC system 700 again prompts 912 the customer for a photo. The process then repeats to decision step 914. Additionally, the VSBC system 700 can optionally transmit the cancelled attempt information to an immutable ledger (e.g., a blockchain) thus alert all the users located on the blockchain that an attempted fraud may have occurred.

If, instead, the verification passes, the VSBC system 700 produces a system prompt 916 that indicates success of the recording and capture process. The VSBC system 700 then forwards 918 the captured data through system bus, volatile memory to Audit Controller for processing, encoding, and permanent recording process. The method 900 then ends.

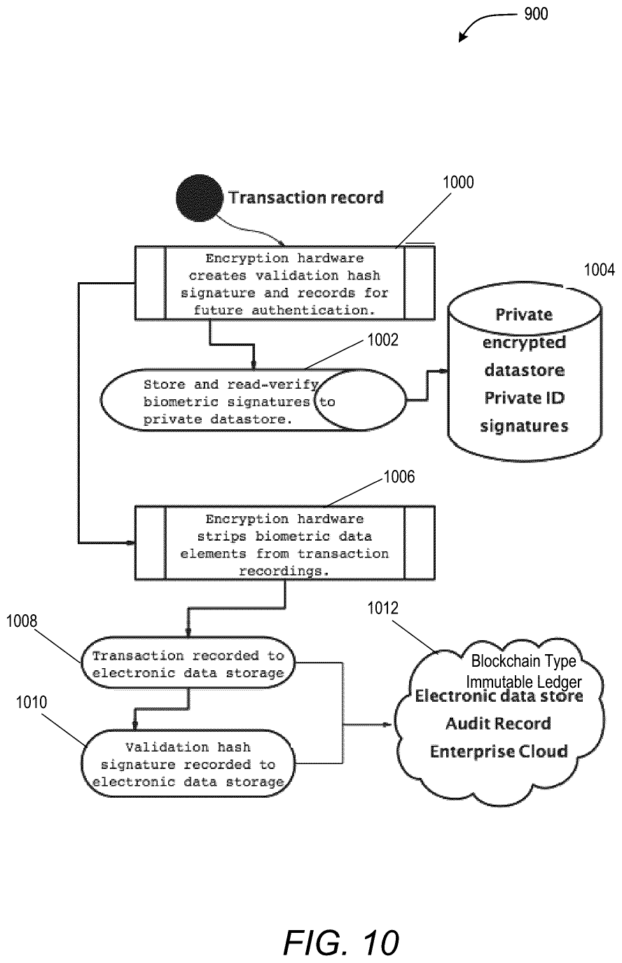

FIG. 10 is a system block diagram of an example of an implementation of a method for exporting audit record to electronic system data stores in Cloud and external applications that meet for example governmental privacy regulations for protection of biometric data in accordance with the present disclosure. In this non-limiting example, the VSBC system, block 1000 includes encryption hardware that creates a validation hash signature and records for further authentication. It utilizes a process that creates a validation hash which is uniquely serial to the captured biometric data recordings. The validation hash provides a permanent record audit trail for chain-of-custody (i.e., extended, and enhanced checksum mathematical function). Block 1002 includes a memory that stores and read-verifies biometric signatures to a private datastore. Block 1002 utilizes a process that outputs a recording to private data stores or database at datastore 1004, and additionally performs a Read-Verify function for a certification or the validation hash described earlier. The datastore 104 is a private encrypted datastore having private identification (ID) signature. This process ensures the integrity of, and private data store retained for future audit capability.

Block 1006 includes encryption hardware that strips the biometric data elements from the transaction recordings. In general, block 1006 provides an encoding and standardization of non-biometric data recordings for delivery to external or Cloud data stores and reporting systems. The data is exported and recorded in block 1008, where the transaction is recorded to an electronic data storage. The data is then passed to block 1010. In block 1010, the validation hash signature is recorded to an electronic data storage. As such, the exported data set include the certified validation hash appended and encoded with the data set. This process excludes any private biometric data from the validation hash and the data set that is not reversible to re-create private biometric data. In block 1012, the VSBC system exports data set to cloud or external immutable ledger or data stores and reporting applications.

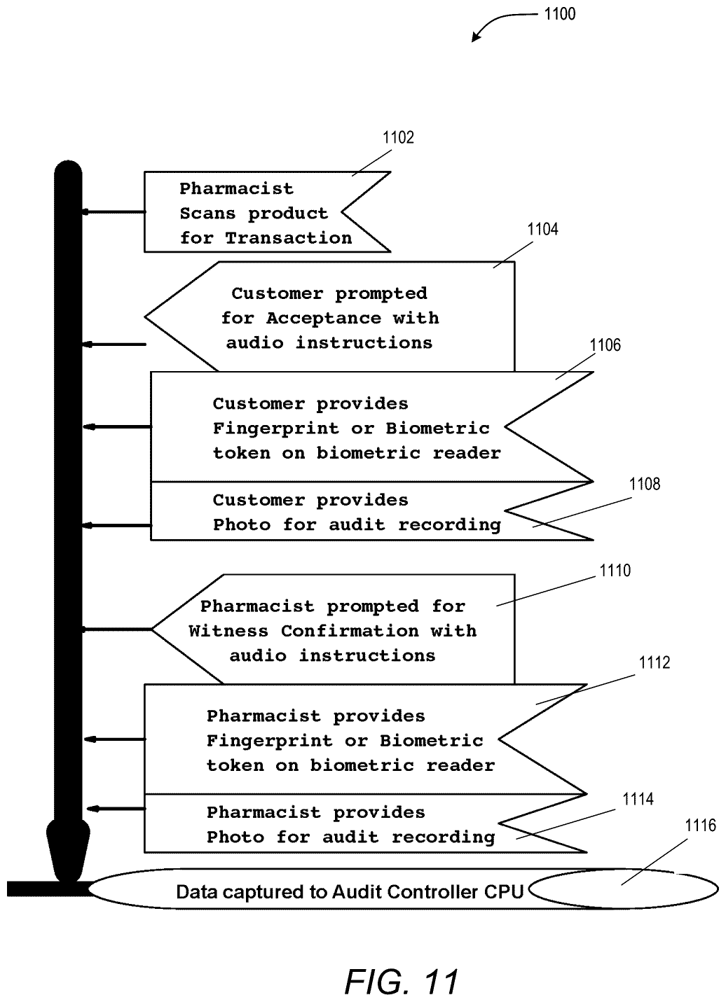

FIG. 11 is a flowchart of an example of an implementation of a method 1100 for data capture in accordance with the present disclosure. In this non-limiting example, the service representative 110 is a pharmacist and the user 112 is a customer at a pharmacy. The method 1100 begins by the pharmacist scanning 1102 a product for a transaction with the customer. The VSBC system then prompts 1104 the customer for acceptance of the transaction with audio instructions. The customer then provides 1106 biometric data to the VSBC system. The biometric data may be biometric recording from the customer indicating an affirmative response, a fingerprint, or biometric token on a biometric reader. The VSBC system then prompts the customer for a photograph and the customer provides 1108 a photograph to accompany the audit recording. The VSBC system then prompts 1110 the pharmacist for a witness confirmation with audio instructions. The pharmacist then provides 1112 a biometric capture that indicates acceptance as a witness of a face-to-face transaction with the customer. The biometric capture may be scanned fingerprint of the pharmacists, or biometric token on a biometric reader. The VSBC system then may prompt the pharmacist for photograph and the pharmacist provides 1114 a photograph as an audio record for audit purposes. In step 1116, the VSBC system aggravates the data and routes it to an audio controller for processing and encoding.

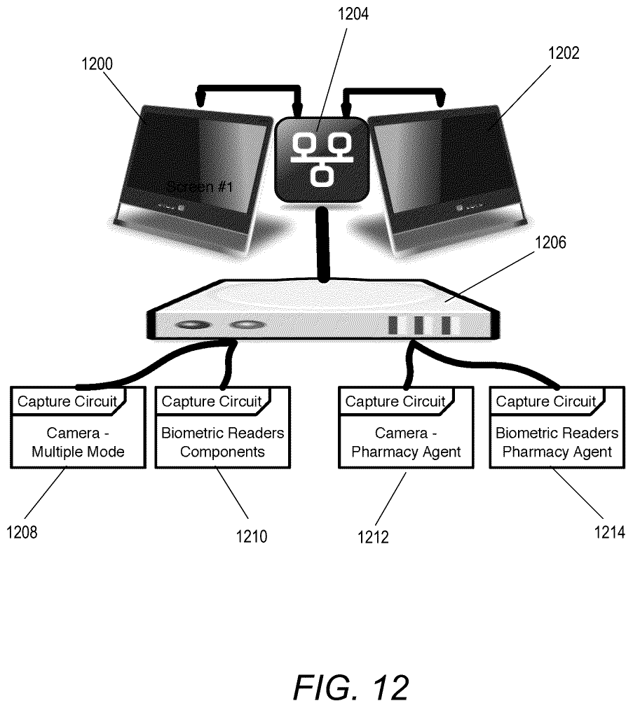

FIG. 12 is a system block diagram of an example of an implementation of hardware logic that may deliver a guaranteed, tamper proof solid-state mirrored visual display with identical data content in accordance with the present disclosure. In this non-limiting example, the VSBC system includes a client video display 1200, vendor video display 1202, control circuit 1204, and second controller 1206. The control circuit 1204 may be a proprietary circuit, component, or device configured to control and recording and verification of the video displays 1200 and 1202. The controller 1206 may be an electronic controller that includes most of the components of the VSBC system that have been discussed earlier. The controller 1206 may be in signal communication with cameras 1208 (for the customer) and 1210 (for the service representative) and biometric readers 1212 (for the customer) and 1214 (for the service representative).

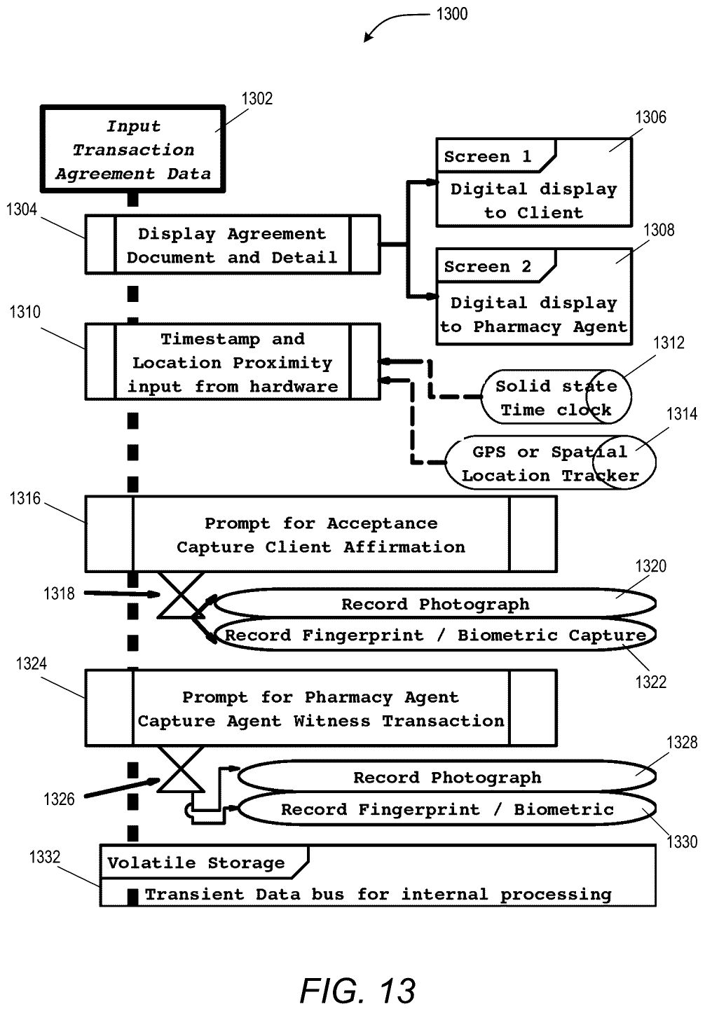

FIG. 13 is a flowchart of an example of an implementation of a data capture method 1300 for recording transactions between two parties while recording physical proximity (i.e., how physically close the two parties are to each other) for audit purpose in accordance with the present disclosure. The method 1300 starts by inputting a transaction agreement data. The data is input from an external source such as, for example, a point-of-sale operating system delivered over a communication bus in signal communication with the VSBC system. In this non-limiting example, the input may originate from various capture devices.

In step 1304, the VSBC system displays the agreement document and details. This may be accomplished through a proprietary video controller where the output may be guaranteed with high assurance to duplicate to identical, matching, and unchanged output to at least two display screens 1306 and 1308 simultaneously. In step 1310, the method 1300 captures a timestamp and a physical proximity proof serialized into the transaction data recordings utilizing a standard solid-state or atomic clock synchronized real time mechanism 1312 and a geo-spatial data source 1314 providing an exact and identifiable location at current time and place that the biometric data was received. It is appreciated by those of ordinary skill in the art that some transactions may be enclosed or underground and, therefore, the said geo-spatial data source may be a governmental-assigned identification, serial number, or certificate in various embodiments. The VSBC system then prompts 1316 the client for acceptance and captures the client's affirmation. In step 1318, the VSBC system utilizes a timed control loop with software control to control the reader devices and record a photograph 1320 or a fingerprint 1322 or other biometric data. The method 1300 then prompts 1324 the service representative (e.g., the pharmacist) to record and capture an agent witness transaction. In step 1326, the VSBC system utilizes a timed control loop with software control to control the reader devices and record a photograph 1328 or a fingerprint 1330 or other biometric data. The method 1300 then captures 1332 aggregated data and sends it to an audit controller for processing and encoding. The method 1300 then ends.

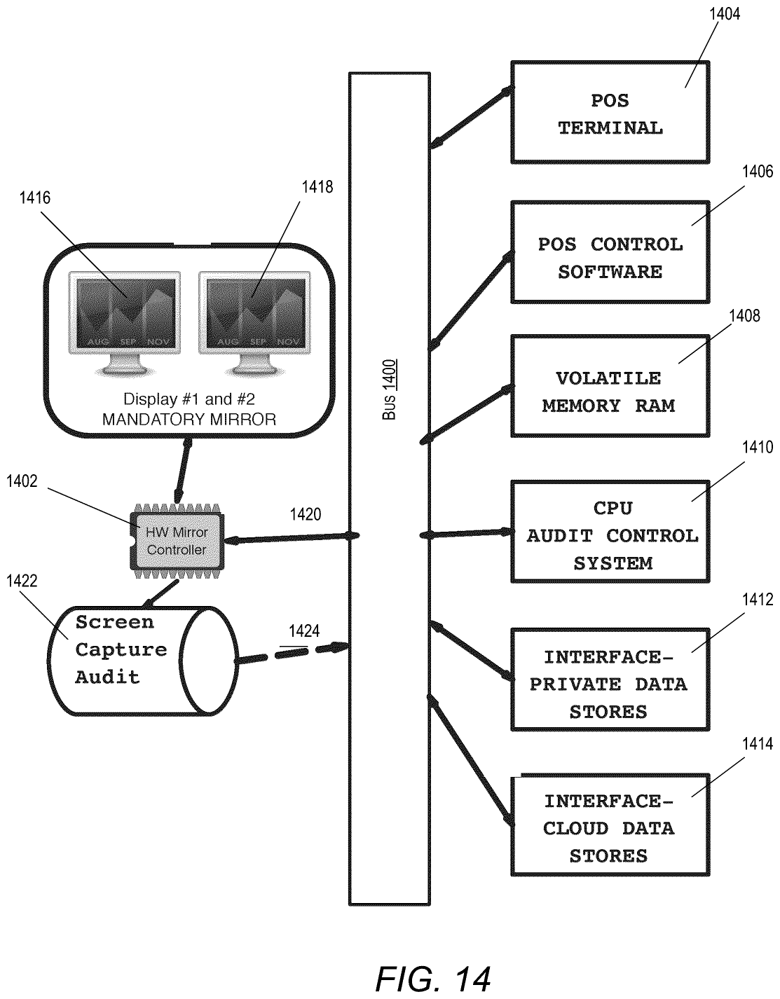

FIG. 14 is a system block diagram of an example of an implementation of the system bus 1400 that provides a certified screen capture audit in accordance with the present disclosure. In this non-limiting example, the bus 1400 is in signal communication with a hardware mirror controller 1402, a point-of-sale (POS) system 1403, POS control software 1406, volatile memory random access memory (RAM) 1408, CPU audit control system 1410, interface-private data stores 1412, and interface cloud data stores 1414.

The hardware mirror controller 1402 may be a proprietary video controller that is configured to guaranteed with high assurance to duplicate to provide an identical, matching, and unchanged output to at least two display screens 1416 and 1418 simultaneously. As an example, the two display screens 1416 and 1418 may be facing opposite each other with 180-degree orientation (so two people can conduct a face-to-face transaction).

In an example of operation, signal 1420 indicates data feed input delivered to the visual controller 1402 from the bus 1400. The VSBC system may include a hardware screen capture device 1422 that is configured to capture recordings at a simultaneous time to be recorded for audit certification where the signal 1424 represents delivery of captured audit data returning to an audit controller system.

In this non-limiting example, the volatile memory RAM 1408 is a volatile data storage mechanism available to audit controller main bus 1400 and the CPU audio control system 1410 is configured to audit control process encompassing control, serialization, data handling and encryption hardware. Furthermore, the interface-private data stores are interfaces to private electronic data stores, for example, private biometric databases for decisions and external biometric computations and the interface-cloud data stores 1414 is an interface to cloud and exported electronic data stores for permanent storage and reporting function externally.

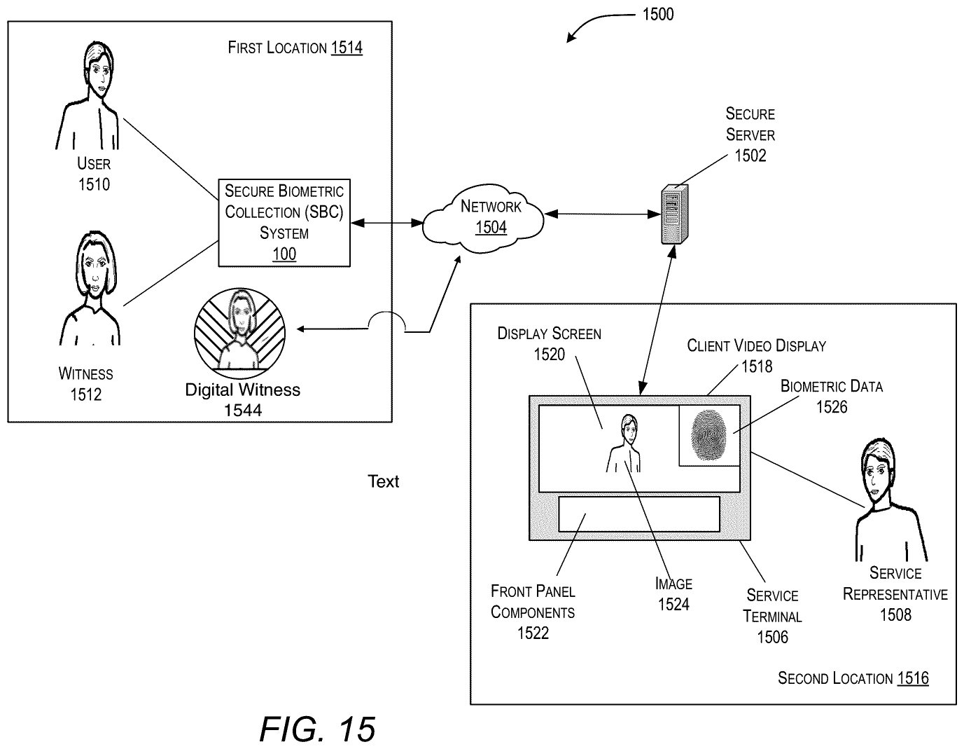

Turning to FIG. 15, a system block diagram of an example of another implementation of the VSBC system 100 within a secure network 1500 in accordance with the present disclosure. Similar to the example described in relation to FIG. 1, the secure network 1502 may include a secure server 1502 that communicates with the VSBC system 100 via a network 1504. Again, the network 1506 may include one or more network such as, for example, an Ethernet network, wireless network, the Internet, a distributed blockchain network or other type of communications network. Again, in this non-limiting example, the service server 1502 may include one or more servers utilized by a business or government entity to communicate and control the VSBC system 100. The secure server 1502 may also communicate with a service terminal 1506 that is utilized by the business or government entity to communicate with the VSBC system 100 via the secure server 1502 and network 1504. Again, the service terminal 1506 may be a computer device, such as a desktop personal computer, laptop computer, or other computer device utilized by a service representative 1508 that works for business or government entity. In this non-limiting example, the secure network 1500 can also be designed as a decentralized blockchain structure.

As before, the secure network 1500 may be a secure network of a business or government entity. The government agency may be an agency that deals with the public that may be for example, taxing agencies, document processing agencies, passport services agencies, a courthouse or court related entities, recording and record keeping government departments, or other similar departments and agencies, etc. Likewise, the business entity may be a health care provider, bank, financial service provider, point-of-service vendor, social media, private enterprise, or other type of business that deals with the public. The government or business entity utilizes the VSBC system 100 to obtain information from a user 1510. As discussed earlier, the user 1510 may be a buyer, customer, patient, or person seeking governmental services or otherwise. The user 1510 may be a person that expects his/her personal information to be kept private. In addition to the user 1510, a witness 1512 (or equivalent AI persona 1544) may be present when the user 1510 interfaces with the VSBC system 100. As a non-limiting example, the witness 1512 may be a parent of the user 1510 if the user 1510 is a minor that is underage. The service representative 1508 may be health provider employee, government employee, or a service representative a vendor for providing services or transactions to the user 1510 that assists the user 1510 in processing his/her needs.

The VSBC system 100, user 1510 and witness 1512 (or AI persona 1544) may be located at a first location 1514 that may be remote from a second location 1516. The service terminal 1506 and service representative 1508 are located at the second location 1516. In FIG. 1, an example was described where the user 112, witness 114, and service representative 110 may be located in the same location, however, in this embodiment, the second location 1516 is located remote from the first location 1514. As an example, the first location 1514 may be within a specific suite of specific floor a building and the second location 1516 may be located either in another suite of the same floor the building, a different floor of the building, or in another building. Moreover, the second location 1516 may be located in another town/city or country from the first location 1514. In all of these examples, the service terminal 1506 is in signal communication with the VSBC system 100 via the network 1504 and secure server 1502.

Furthermore, the second location 1516 of the service terminal 1506 and service representative 1508 may be remote from the location of the secure server 1502, where the second location 1516 may be located in another town/city or country from the location of the secure server 1502 and the secure server 1502 may be in signal communication with the service terminal 1506 via another network (not shown) that again includes one or more network such as, for example, an Ethernet network, wireless network, the Internet, a distributed blockchain network or other type of communications network.

In a telemedicine type of example, the VSBC system 100 may be a standalone kiosk in a medical facility at the first location 1514 and the service representative 1508 and service terminal 1506 are located a second location 1516 that may be in another building remote from the medical facility. Alternatively, in a telebanking type of example, the VSBC system 100 may be a standalone kiosk in a virtual bank (i.e., a bank facility without bank tellers or personnel) at the first location 1514 and the service representative 1508 and service terminal 1506 are located a second location 1516 that may be in another building remote from the virtual bank. It is appreciated that similar examples can be described for point-of-service commercial facilities or government facilities.

In these examples, the service terminal 1506 may include a client video display 1518 having a display screen 1520 and front panel of components 1522. As a result, the service representative 1508 can see an image (s) 1524 of a photograph (s) and/or video of the user 1510 on the display screen 1520. These image (s) 1524 are recorded by the one or more cameras on the VSBC system 100. Also, once the user 1510 provides their biometric data (as described earlier such as, for example, a scanned fingerprint) to the VSBC system 100, the VSBC system 100 may be configured to transmit that biometric data to the service terminal 1506 that displays it as biometric data 1526 on the display screen 1520. In response to this information, the service representative may provide their own biometric data (such as, for example, a scanned fingerprint) to the VSBC system 100 via a biometric input device (such as, for example, a fingerprint scanner) at front panel components 1522 of the service terminal 1506.

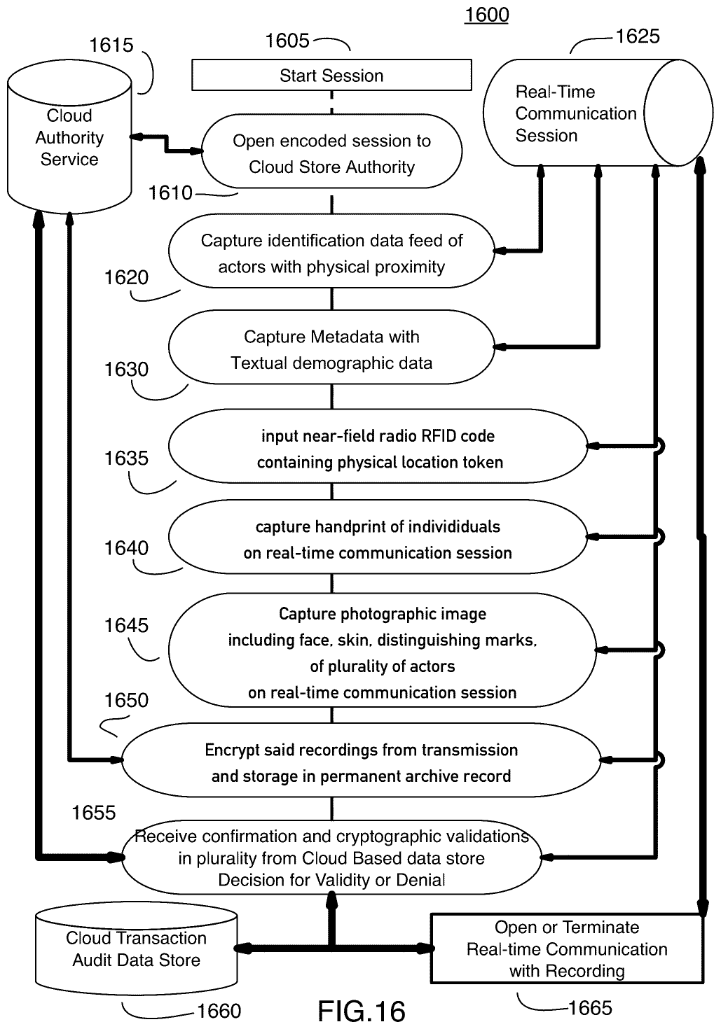

FIG. 16 is a flow diagram 1600 of one example implementation steps of the exemplary VSBC system with external Software-as-a-service (Saas) Cloud system/service 1615. As a non-limiting example, a session hosted or “gated” by the VSBC system would have a session start 1605 which would open an encoded session to Cloud Store Authority 1610, which manages connection to a Cloud Authority Service 1615, wherein encoded data from the VSBC session would be stored. Next, some form of identification capture of the user (s) (actor) with consideration of physical proximity would be taken 1620 via a real-time communication device 1625. If an AI persona is utilized as a witness (not shown), data from the AI persona would be passed into the VSBC system to verify the “humanness” or identity of the user in one or more of the identification steps in this session. Next, metadata with textual demographic data such as Party names, locations, contacts, credentials, account numbers, and organization authority data, would be captured 1630, if desired, via real-time communication device 1625. A location determination via an RFID can be next obtained via reading an RFID code containing a physical location token 1635, using real-time communication device 1625. Or a scanning of Q-code or the like physically co-located.

A biometric input, for example, handprints, fingerprints, etc. of the user (s) could next be input 1640, via real-time communication device 1625, either directly (the biometric sensor is a physical part of the real-time communication device 1625 or the biometric sensor is a separate physical device which communicates with the real-time communication device 1625, using it as a gateway to the VSBC system). Likewise, image capture would next be acquired 1645 including one or more official features, skin, distinguishing marks, tattoos, etc. of the user (s)/actor (s) interfacing with the VSBC system via real-time communication device 1625. Recorded data from these exchanges and inputs would be received via real-time communication device 1625 to be encrypted 1650 and transmitted for storage in a permanent archive record, for example a blockchain or other permanent storage solution in the Cloud Authority Service 1615. Receipt of confirmation and cryptographic validation 1655 would next be evaluated to determine if the Cloud Authority Service 1615 did indeed properly store the transmitted encrypted data. If denied, the process would proceed to Open or Terminate real-time communication with Recording 1665 to either terminate the session after a predetermined number of denials, or re-open the session for another attempt at validating the user (s) (actors) via real-time communication device 1625, restarting at either step 1610 or 1620. Cloud Transition Audit Data Store 1660 serves to convert data from the session into a cryptographic hash Audit Token data element, containing for example, transaction metadata, identification recordings, and verification of identification recordings, which is then encrypted into an Audit Token. The encrypted Audit Token can be sent to the Cloud Store Authority 1610 to access the Cloud Store Authority 1610. In some embodiments, for ease of data transfer, the Audit Token and a common-format PDF Receipt can be delivered to external systems for confirmation and recording of a transaction.

Illustrating an example of the embodiment FIG. 16 as applied into Social Media real-time communication, consider this flow for this simple application, understanding variations and added security components may be added. VSBC™ system initiates an electronic communication session at Step 1605, which activates with an encoded session to VSBC™ Cloud Authority Service 1610, where 1610 is the authoritative trusted controller for Activating and Deactivating relevant social media communication, next Step 1620 opens and captures user identification and physical proximity through Real-Time communication session 1625, which can be through varied mediums and styles of communication. Step 1630 is mandatory capture for meta-data or technical data associated with session 1625. Step 1635 illustrates capture of physical location with an RFID reader, for example, and Step 1640 illustrates recording of Fingerprint/Handprint/Biometrics from industry standard reader hardware (e.g., Live scan or non-Live scan optical readers). Step 1640 includes physical location metadata compliant with commercial or industry standards and Step 1645 captures visual identification data from photographic reader hardware. Step 1650 accumulates all said recordings with filtering and redaction function retaining selected data into an encrypted permanent record delivered to VSBC™ Cloud Authority Service 1610, in certain embodiments. Step 1655 indicates a Decision State from Cloud Authority Service 1610 which delivers control action for Activation, Denial Termination, and retain current open state. Transaction results data are recorded into Cloud Audit block 1660, which can be a transparent block-chain storage in ideal embodiments, and other variations for permanent storage optional. Final closure Step 1665 illustrates the termination and triggering final audit recording data and meta data into Cloud Audit block 1660.

The exemplary system, in various embodiments, can transmit a permanent, time stamped and geographically-stamped Audit Token to external public cloud archive data stores, when geographic stamping contains GPS, GLONASS, and terrestrial cellular Location Data. As stated in prior embodiments, a wipe of acquired or recorded data can also be achieved, for example, via a cryptographic sanitize (non-recoverable erase). Variations of the described system can be where the encoded data recordings are coded into a witness and notary transaction, a certificate document passed to whatever transaction destination is being accessed to have encrypted identity tokens. Transaction details can be encoded into non-repudiable, one-time transaction encoding which contains identity tokens in plurality. The exemplary system can transmit a common-format PDF Document certificate into Cloud Based Data store, containing serialized confirmation specifying the Identity and transaction token recordings, for future verification application.

It is expressly understood the various steps outlined above may be performed in an order that is different from that shown and some steps may be skipped or merged into other steps, depending on the implementation and objectives of the commercial embodiment chosen. And that additional steps may be instituted. If a step fails to verify or the data acquired/input is determined to be faulty, the respective step may request the user (s) to resubmit their data (or biometric) for a re-verification. In some embodiments, if present, the AI persona (not shown) could offer the resubmission request to the user or provide biometric calibration instructions to the user. In some embodiments, the AI person may be the primary means of instructing the user's interactions and manage proper interfacing with the hardware sensors or data input to the VSBC system. This would be not very much different than the example of having a “live technician” (the AI persona proxying as such) assist a patient (user) in self-application of medical equipment for measuring the health of the patient (user). Thus, the role of the AI persona can also be to facilitate the user's interactions with the VSBC system as well as to act as a digital witness of the user.

In the input steps outline above, it is understood that a large suite of multiple biometrics or user-input data choices could be available and only a subset of such from the user (or biometric selection) could be requested, to arrive at a sufficient level of verification. For example, a fingerprint scan may prove unverifiable wherein the system could alternately request an eye scan as a substitute. Or, the user is not able to provide or chooses to not provide the requested action/measurement, then the system would offer another option.

FIG. 17 is a block/function diagram 1700 of an VSBC system showing an example of a digital electronic witness or AI persona 1702 being utilized as a 3^{rd} party witness or adjunct for the human witness 1512, in concert with computational exchange and/or storage with a Cloud 1750. Cloud 1750 may be analogous to the Cloud Authority Service described in the embodiment of FIG. 16, wherein the described session management steps are utilized, if so desired. It should be noted that in some implementations, human witness 1512 (if present) may be used as a “partial” witness to perform certain verification tasks that the AI persona 1702 cannot (or not efficiently) perform, or the human witness 1512 may act as a proctor for the exchanges between the AI persona 1702 and the user 1510, as well as confirming the conclusions or observations made by the AI persona 1702. That is, the human witness 1512 may assist the AI persona 1710. Thus, depending on the sophistication of AI persona 1702, human witness 1512 may be present or not present, and if present may have a collaborating role with the AI persona 1702. Aspects of the user 1510 and witness 1512 are similar to those described in the previous embodiments and are not restated here.

Generally in one speaking, embodiment, Block 1710 represents a suite of options the exemplary configuration can use when interacting with the VSBC system 100 and/or the user 1510 and/or the witness 1512. In this example, the electronic witness (or AI persona) 1702, can monitor or proctor the exchanges with the user 1510. If designated as a proxy to the human witness 1512, AI persona 1702 will be given access to information between the VSBC system 100 and the user 1512, so as to provide its assessment of the identity and/or verification of humanness of the user 1512. With this “in-line” configuration, the AI persona 1702 can also act as quality control, reviewing the input information from the VSBC system 100 and decide if additional metrics from the user 1512 is needed, for user identification or for user as-a-human verification. For the former, the capability of determining whether the user is who he says he is, can be offloaded to an AI engine who can evaluate the input data and make an AI-based user identification determination. Thus, the AI persona 1702, in this implementation acts as the identification logic. Conversely, the AI persona 1702 can utilize the hardware in the VSBC system 100 to perform human-user verification, evaluating the data stream from the VSBC system 100 and apply AI to determine if the user is human. Thus, rather than having separate hardware from the VSBC system 100, the AI persona 1702 can utilize the hardware already in the VSBC system 100.

Whether the AI persona 1702 acts as the “transaction QC monitor” or as a substitute digital witness, another option in block 1710 (containing steps 1702-1708) is recording 1704 the user's actions and data input by the user so as to provide an “identity” record of the session. Documents or information 1706 to interface or solicit a response from the user 1510 can be fed by the AI persona 1702 or separately into the VSBC system 100, to obtain identity verification and to obtain human verification of the user 1510 (if AI persona 1702 is tasked to do so). If the AI persona 1702 is monitoring the session record 1704 and documents 1706, it can assist in determining if the user is not an imposter, or to confirm user-data from the VSBC system 100 is not hacked information from an impostering electronic source. These documents can include information regarding the user's 1510 social security no., birth date, account no., prior residences, family history, income, credit, etc. Submission ordering of the documents to the user 1510 can be via a probability metric or other metric, determined by the system. Responses by the user 1510 can then be audited for identity determination and/or user verification 1708 via confirmation from physical witness 1512 or via virtual witness (e.g., AI persona) 1702. As alluded above, these steps may be controlled by the AI persona 1702 or separately.

A variation of an implementation of electronic witness 1702 is also shown in the embodiment of FIG. 17, wherein a computational, probability scoring method can be included in the VSBC capture system. This variation can be applied or deployed with a plethora of formats and mathematical rules. In this example, electronic witness 1702 represents a computational probability scoring witness, where step 1704 implements identity recording methods. Step 1706 indicates the input for document inputs with probability scoring data elements, which can be various algorithm formats proprietary and standardized (such as Fingerprint matching algorithms for a common example), and Step 1708 can indicate high-performance high complexity audit scoring decision calculations with, for example, deterministic probability varied algorithms, proprietary and/or standardized. One can observe that different embodiments might employ wildly-varied algorithm types, styles and electronic computational chip types in the performance of this Step 1708.

In a workflow example, the following supporting steps occur. Step 1730 initiates with an input trigger from identity data, Step 1732 processes calculations based upon biometric data stores into actionable probability lookup tables, further performing aggregation and cross-checking from a plurality of probability lookup tables multi-modality, and upon “more than one” modality. In lay terms, this means multiple identifiers such as FBI palm prints, eye scoring, facial bone feature, tattoo and body art notations, etc. These are non-limiting examples to help clarify Step 1732. Step 1734 is an administrative mandatory chain-of-custody recording for time and date encoding with non-tamperable hashing (e.g., checksum type cryptography with various algorithms), Step 1736 records the authority or source of authority that records into the audit record for future transparency and review, including scoring results, deviations (allowable discrepancy margins for calculating error), and raw data for replay and re-calculation. Resulting in Step 1738 which is a documented and proven decision step for Acceptance or Denial integrated back to Block 1710.

To describe further examples or embodiments, various steps shown in block 1710 can work hand-in-hand with supporting steps 1730-1738. For example, prior to or at some point in the session, the identity determination audit 1708 concludes the “witness” confirmation is below a threshold for acceptance, the system may restart the session or terminate. In some modes, if accepted, then notarizing 1736 the events and transactions in the session can occur. Image, data capture, timestamping, encoding, biometric recording and so forth 1734 can be done, wherein step 1732 calculates from the received data in step 1734 a metric for determining whether the user is who he says he is. If the user is identified with sufficient confidence or a threshold is met for identity verification, an identity-token 1730 is triggered. The trigger 1730 is an indication that the user has validated himself and the system can proceed with the desired transaction. Communication of the triggering token 1730 can be moderated via block 1710's steps to access VSBC system 100's communication channel with Cloud 1750. Here, Cloud 1750 may be a storage Cloud, a government entity, document processing agency, court services, health care provider, bank, financial service provider, point-of-service vendor, social media, private enterprise, or any other type of service being supported by Cloud 1750.

As apparent from the various embodiments described above, the exemplary system can be configured to operate with cloud-based data storage with a live encoded interface. The exemplary system also enables the “capturing” of at least two “persons” in a single unified transaction for purposes of proving physical proximity and spatial geographic-location of the two “persons.” Therefore, the interaction of the two locally confirmed “persons,” produces a robustness against a single imposter or malicious software acting as an imposter. To facilitate security of the user, the system can devise an identification related visual token code, capture visual and non-visual (IR) images of the user and user's features. Data between the VSBC and external devices and hardware or Cloud can be protected via internally initiated or externally initiated preemptive encryption prior to transmission. Similarly, transactions and recordings of a session can be preemptively encrypted.

It is understood that one or more of the steps states above and variations thereof can be encoded into software as modules or submodules, being run by a processor. Therefore, enablement of various steps as software or algorithms operating in tandem with hardware devices, sensors, processors and the like are within the capabilities of one of ordinary skill in the art and within the spirit and scope of this disclosure.

Conditional language such as, among others, “can,” “could,” “might” or “may,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example.

It will also be understood that various aspects or details of the invention may be changed without departing from the scope of the invention. It is not exhaustive and does not limit the claimed inventions to the precise form disclosed. The description of the different examples of implementations has been presented for purposes of illustration and description, and is not intended to be exhaustive or limited to the examples in the form disclosed. Many modifications and variations will be apparent to those of ordinary skill in the art. Further, different examples of implementations may provide different features as compared to other desirable examples. The example, or examples, selected are chosen and described in order to best explain the principles of the examples, the practical application, and to enable others of ordinary skill in the art to understand the disclosure for various examples with various modifications as are suited to the particular use contemplated. The claims and their equivalents define the scope of the invention.

1. A verified secure biometric collection system for collecting and recording data from a user associated with a secure transaction, comprising:
a storage device;
an input interface configured to receive information from the user related to the secure transaction;
a camera configured to capture an image of the user;
a biometric scanner configured to scan a biometric information of the user;
a camera encoder configured to combine and hash the image of the user into combined data sets that are stored in the storage device;
a biometric encoder configured to process the biometric scan of the user into biometric template data sets and store the biometric template data sets in the storage device;
at least one of a human witness and non-human digital witness having access to sensor inputs and software, configured to verify that the user providing at least one of the image capture and biometric scan is the user, wherein the at least one human witness and non-human digital witness is in proximity to the user; and
a privacy encoder configured to combine and encrypt the combined data sets and biometric template data sets from the storage device into encrypted data sets memorializing the secure transaction,
wherein the system is configured to produce a copy of the secure transaction that includes the encrypted data sets for the user, and
wherein the storage device is configured to be purged of all the data collected from the user including the information from the user and all biometric data that includes the image of the user, biometrics of the user, combined data sets, biometrics template data sets, and encrypted data sets once the copy of the secure transaction is produced.

2. The system of claim 1, further including a second camera, wherein
the first camera is configured to capture the image of the user in a visible range,
the second camera is configured to capture a second image of the user in a non-visible range, and
the camera encoder is configured to combine and hash the image of the user in the visible range and the image of user in the non-visible range into the combined data sets.

3. The system of claim 1, wherein the system is further configured to transmit the data collected from the user including the information from the user and all biometric data that includes the image of the user, the biometric measurement of the user, combined data sets, biometric template data sets, and encrypted data sets once the copy of the secure transaction is produced to an immutable distributed ledger upon receiving the user's instruction.

4. The system of claim 1, wherein the sensor inputs are one or more witness-cameras in proximity to the user, having image processing software to assist in the verification.

5. The system of claim 4, wherein the sensor inputs further comprises, at least one of a microphone, speaker, thermal sensor, and display, with additional processing software to assist in the verification.

6. The system of claim 1, wherein the non-human digital witness comprises an artificial intelligence engine, operating to analyze sensor data taken of the user to verify the user is an actual person.

7. The system of claim 6, further comprising at least one of an avatar and robotic image as an interface for the artificial intelligence engine with the user.

8. The system of claim 6, wherein the artificial intelligence engine works in concert with data from at least one of the input interface, camera, and biometric scanner for verification of the user.

9. The system of claim 1, further including a processor, wherein
the biometric encoder is further configured to timestamp the biometric scan of the user with a first timestamp and a verification indication from the non-human digital witness with a second timestamp, and
the processor is configured to determine a difference between the first timestamp and second timestamp is less than or equal to a predetermined time.

10. The system of claim 1, wherein the storage device is in a network Cloud and the input interface operates as a live encoded interface.

11. The system of claim 1, further comprising, a visual token code produced from the privacy encoder and assigned to one or more captured images of the user.

12. The system of claim 1, further comprising:
a cryptographic hash Audit Token data element, the data element containing at least one of transaction metadata, identification recordings, and verification of identification recordings; and
an encrypted Audit Token from encrypting the data element, wherein the encrypted Audit Token provides access to a connected Cloud-based data store.

13. The system of claim 1, further comprising, an Audit Token and a common-format PDF Receipt, wherein the Audit Token is used to inform an external system of confirmation and recording of the transaction.

14. The system of claim 1, further comprising, a certificate document generated from the transaction, containing an encrypted user identity token.

15. The system of claim 1, further comprising, a non-repudiable, one-time transaction encoding of details of the transaction, containing user identity tokens in plurality for a plurality of users.

16. The system of claim 1, wherein said user is at least one of a human or artificial intelligence created persona.

17. A method of collecting and recording data from a user associated with a secure transaction, comprising:
receiving information from a user via an input interface, relating to the secure transaction;
capturing an image of the user;
scanning biometric information of the user;
electronically combining and hashing the image of the user into combined data sets for storage;
electronically processing the biometric information scan of the user into biometric template data sets for storage;
witnessing the user via at least one of a human witness and non-human digital witness, in proximity to the user;
providing a verification of the user via the witnessing;
electronically combining and encrypting the combined data sets and biometric template data sets from storage into encrypted data sets memorializing the secure transaction;
electronically producing a private copy of the secure transaction including the encrypted data sets for the user; and
purging from storage all the data collected from the user including the information from the user and all biometric data that including the image of the user, biometric information of the user, combined data sets, biometric template data sets, and encrypted data sets once the copy of the secure transaction is produced.

18. The method of claim 17, further comprising, capturing an image of the user in a non-visible range, wherein image data of the non-visible range is also combined and hashed into the combined data sets.

19. The method of claim 17, wherein the system is further configured to transmit the data collected from the user including the information from the user and all biometric data that includes the image of the user, the biometric measurement of the user, combined data sets, biometric template data sets, and encrypted data sets once the copy of the secure transaction is produced to an immutable distributed ledger upon receiving the user's instruction.

20. The method of claim 17, further comprising, providing one or more witness-cameras in proximity to the user to assist in the verification by the non-human digital witness.

21. The method of claim 20, further comprising, acquiring real-time data of the user via at least one of a microphone, speaker, thermal sensor, and display,

22. The method of claim 17, further comprising, using an artificial intelligence engine as the non-human digital witness.

23. The method of claim 22, further comprising, interfacing with the user via at least one of an avatar and robotic image.

24. The method of claim 22, further comprising, processing data from the captured image of the user and the scanned biometric information of the user by the artificial intelligence engine for verification of the user.

25. The method of claim 17, further comprising:
timestamping the biometric scan of the user with a first timestamp and a verification indication from the non-human digital witness with a second timestamp; and
determining a difference between the first timestamp and second timestamp is less than or equal to a predetermined time.

26. The method of claim 17, further comprising, producing a visual token code and assigning to one or more captured images of the user.

27. The method of claim 17, further comprising, cryptographically hashing an Audit Token data element containing at least one of transaction metadata, identification recordings, and verification of identification recordings, to form an encrypted Audit Token for accessing a Cloud-based data store.

28. The method of claim 17, further comprising, informing an external system of confirmation and recording of the transaction via an Audit Token and a common-format PDF Receipt.

29. The method of claim 17, further comprising, generating a certificate document from the transaction, containing an encrypted identity token.

30. The method of claim 17, further comprising, encoding details of the transaction in a non-repudiable, one-time transaction format, containing identity tokens in plurality for a plurality of users.

31. The method of claim 17, further comprising, upon completing the secure transaction, enabling access for the user to at least one of a real-time communication session, social media session, restricted access session, government interaction session, financial transaction session, and virtual world session.

32. The method of claim 17, further comprising, enabling at least one of transaction certification, transaction certification, and recording of encrypted transaction tokens.

33. The method of claim 17, further comprising, electronically notarizing, via the non-human digital witness, the transaction.

34. The method of claim 17, further comprising, proving physical proximity and spatial geographic-location of the user to the input interface.

35. The method of claim 17, further comprising, reading and recording biometric data for a plurality of human and non-human digital witnesses proximal to the user input.

36. The method of claim 17, further comprising, proving physical proximity and spatial geographic-location of the user to the input interface.

37. The method of claim 17, further comprising, capturing via a non-visible light capturing camera at least one of facial, skin, and distinguishing marks of the user.

38. The method of claim 17, further comprising, pre-emptively encrypting and privatizing recordings for real-time transmission and storage in a Cloud-based data store.

39. The method of claim 38, further comprising, receiving confirmation and cryptographic validations, in plurality, from the Cloud-based data store, to determine validity or denial of validity for the recordings.

40. The method of claim 36, further comprising, transmitting a permanent, timestamped and geographically-stamped Audit Token to an external public cloud archive data store, when geographic location contains at least one of GPS, GLONASS, and terrestrial cellular location data.

41. The method of claim 17, further comprising, electronically performing a cryptographic non-recoverable erase on data recordings.

42. The method of claim 17, further comprising, transmitting at least the encrypted data sets and secure transaction into at least one of a Cloud Based Auditing service, Cloud Based recording service, third-party computing system and governmental computing system.

43. The method of claim 17, further comprising, transmitting a common-format PDF Document certificate into a Cloud Based Data store, containing serialized confirmation specifying user identity and transaction token recordings, for future verification application.

44. The method of claim 17, wherein said user is a human or artificially intelligent persona.

45. A verified secure biometric collection system for collecting and recording data from a user associated with a secure transaction, comprising:
a storage device;
an input interface configured to receive information from the user related to the secure transaction;
a reader configured to capture data information of the secure transaction;
a biometric scanner configured to scan biometric information of the user;
a camera encoder configured to combine and hash the data of the secure transaction into combined data sets that are stored in the storage device;
a biometric encoder configured to process the biometric scan of the user into biometric template data sets and store the biometric template data sets in the storage device;
at least one of a human witness and non-human digital witness, having access to sensor inputs and software, configured to verify that the user providing the data and biometric scan is the user, wherein the at least one human witness and non-human digital witness is in proximity to the user; and
a privacy encoder configured to combine and encrypt the combined data sets and biometric template data sets from the storage device into encrypted data sets memorializing the secure transaction,
wherein the system is configured to produce a copy of the secure transaction that includes the encrypted data sets for the user, and
wherein the storage device is configured to be purged of all the data collected from the user including the information from the user and all biometric data that includes the data information, biometrics of the user, combined data sets, biometrics template data sets, and encrypted data sets, once the copy of the secure transaction is produced.

46. The system of claim 45, wherein the system is further configured to transmit the data collected from the user including information and all biometric data that includes the biometric measurement of the user, combined data sets, biometric template data sets, and encrypted data sets once the copy of the secure transaction is produced to an immutable distributed ledger upon receiving the user's instruction.

47. The system of claim 45, further comprising, at least one of a credit card, a debit card, a digital passport card, a bank access card, a stored value monetary card, and virtual identity token, providing at least one of a physical and digital identification,
wherein the sensor inputs are one or more reader (s) in proximity to the user, wherein the at least one physical card is embedded with processing software to assist in the verification.

48. The system of claim 45, wherein the non-human digital witness comprises an artificial intelligence engine operating to analyze sensor data taken of the user to verify the user is an actual person.

49. The system of claim 48, further comprising at least one of an avatar and robotic image as an interface for the artificial intelligence engine with the user.

50. The system of claim 48, wherein the artificial intelligence engine is configured to work in concert with data from at least one of the input interface, reader, and biometric scanner for verification of the user.
